(19) United States
(12) Patent Application Publication
(10) Pub. No.: US 2025/0011362 A1
(43) Pub. Date: Jan. 9, 2025

(54) COMPOUNDS AND COMPOSITIONS THEREOF FOR THE TREATMENT OF CANCER

(71) Applicant: Clarity Pharmaceuticals Ltd, Eveleigh (AU)

(72) Inventors: Lachlan Eion MCINNES, Eveleigh (AU); Matthew John HARRIS, Eveleigh (AU); Ellen Marianne VAN DAM, Eveleigh (AU); Colin BIGGIN, Eveleigh (AU)

(21) Appl. No.: 18/713,134

(22) PCT Filed: Nov. 24, 2022
(86) PCT No.: PCT/AU2022/051410

§ 371 Date: May 23, 2024

(30) Foreign Application Priority Data
Nov. 24, 2021 (AU) 2021903792

(51) Int. Cl. C07K 5/107; A61K 51/08

(52) U.S. Cl. CPC C07K 5/1016; A61K 51/088

(57) ABSTRACT

The present invention relates to compounds comprising a metal chelator and two fragments capable of binding to PSMA, compositions thereof and uses thereof in methods of treatment.

### FIELD OF THE INVENTION

The present invention relates to compounds comprising a metal chelator and two fragments capable of binding to PSMA, compositions thereof and uses thereof in methods of treatment.

### BACKGROUND

Prostate cancer is a leading cause of cancer-related deaths in men and is currently treated using one or more techniques selected from surgery, radiotherapy, chemotherapy and hormone therapy. Although there are a variety of potential avenues for treatment, not all options may be suitable for a given patient or once administered, not all treatments may be successful.

Drawbacks of treatment options such as radiotherapy and chemotherapy include the potential for unwanted side effects to be experienced by the patient, often owing to the limited specificity a particular compound may have for the target cancer site. This means that in addition to treatment that is less efficient, the patient may need to undergo a therapy regime that is accompanied by additional discomfort. Furthermore, damage of healthy tissue may occur, again owing to the administered therapy acting at sites that are not the target cancer site.

It is known that prostate cancer is often characterised by overexpression of prostate-specific membrane antigen (PSMA), however the ability for an administered compound to target cancer sites in vivo that overexpress the target antigen (in the presence of any naturally expressed antigen) is often limited. Furthermore, even where a radiotherapy or chemotherapy agent does show the ability to target PSMA specifically, it must also have the appropriate retention and metabolism characteristics.

For the purposes of treating prostate cancer by identifying and targeting sites that overexpress PSMA, one approach to increasing the binding of a given compound to a cancer site is to increase the parts of the compound that target PSMA. This may include increasing the number of moieties in the compound that target PSMA, however this in turn results in a larger compound such that characteristics such as solubility, retention and metabolism (all of which may be linked to molecular weight) are also modified and potentially modified unfavourably. For example, the compound must be soluble under physiological conditions, be transported in the circulatory system, have sufficient binding at the target site and have a favourable metabolic and toxicity profile (e.g. minimal accumulation in the liver to limit hepatotoxicity, minimal accumulation in the kidneys to limit nephrotoxicity).

Furthermore, specifically in the cases of agents for radiotherapy (i.e. radiopharmaceuticals), the compound must also have the ability to coordinate, retain and deliver a suitable radionuclide to the cancer site.

There exists a need for compounds that can provide improved binding affinity to cancer sites expressing PSMA and have the ability to provide radiotherapeutic and radioimaging properties to the intended site. There is also a requirement that the compounds are sufficiently stable and do not undergo decomposition during use, but also undergo metabolism and elimination after a certain time.

### SUMMARY OF THE INVENTION

The compounds of the present invention contain a metal chelator and two lysine-urea-glutamic (Lys-urea-Glu) acid moieties each attached to the metal chelator by separate linkers. The Lys-urea-Glu moiety binds prostate-specific membrane antigen (PSMA), which is overexpressed on the surfaces of some cancers, for example, prostate cancer. The linker binding the Lys-urea-Glu moieties to the metal chelator comprise two alkylene chains joined by two phenylalanine residues, with various intervening amide linkages joining these groups together. The alkylene linkers and phenylalanine residues linking the Lys-urea-Glu moieties with the metal chelator act to separate these two latter groups. This ensures that the activity of the Lys-urea-Glu moieties and the metal chelator do not interfere with each other. However, it is important that the separation between the metal chelator and the Lys-urea-Glu moieties are not separated by a distance such that the metal chelator cannot deliver the metal contained within to the site that is targeted by the Lys-urea-Glu moieties. The present inventors have found that the compounds of Formula (I) disclosed herein comprising two moieties that are capable of binding to PSMA allow for more efficient methods of radioimaging and treatment where compounds of Formula (I) complexed with a radionuclide are administered to the subject.

In contrast to compounds containing a single Lys-urea-Glu moiety, the present inventors have found that compounds disclosed herein show increased binding and retention at sites expressing PSMA, i.e. a greater proportion of the administered compound is bound and retained at the desired site. This in turn leads to more efficient treatment and imaging of a cancer that is associated with the overexpression of PSMA. Since the compounds disclosed herein show better binding to the target site, then a smaller or larger dose of the compound can be administered to the subject as required. For example, as the compounds disclosed herein are capable of chelating a suitable radionuclide, this then means that a smaller dose of radiation can be administered to the subject, while still providing images, or a larger dose of radiation can be administered to the subject for treatment of a requisite standard. Where a smaller dose of radiation is administered, this reduces the likelihood and/or severity of any unwanted side effects that arise due to the administration of a radionuclide, i.e. off-target radiation damage.

Since the compounds of the present invention contain a radionuclide for radioimaging and radiotherapy, the compounds must be retained for a sufficient time for imaging or therapy, however the compounds must also be metabolised and excreted from the subject after a time. The present inventors have found that even though the compounds of the invention containing two PSMA-binding moieties and therefore have a greater molecular weight, the compounds show improved binding and retention so as to provide better images during radioimaging and retention at the cancer site expressing PSMA, the compounds also show the requisite stability and physical properties for metabolism in a desired timeframe.

In a first aspect, the present invention provides a compound of Formula (I):

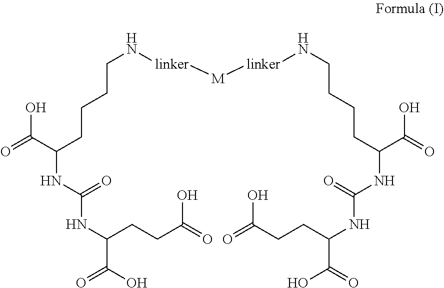

or a pharmaceutically acceptable salt, complex, isomer, solvate or prodrug thereof,
wherein each linker may be the same or different;
wherein M is a residue of a metal chelator selected from the group consisting of:

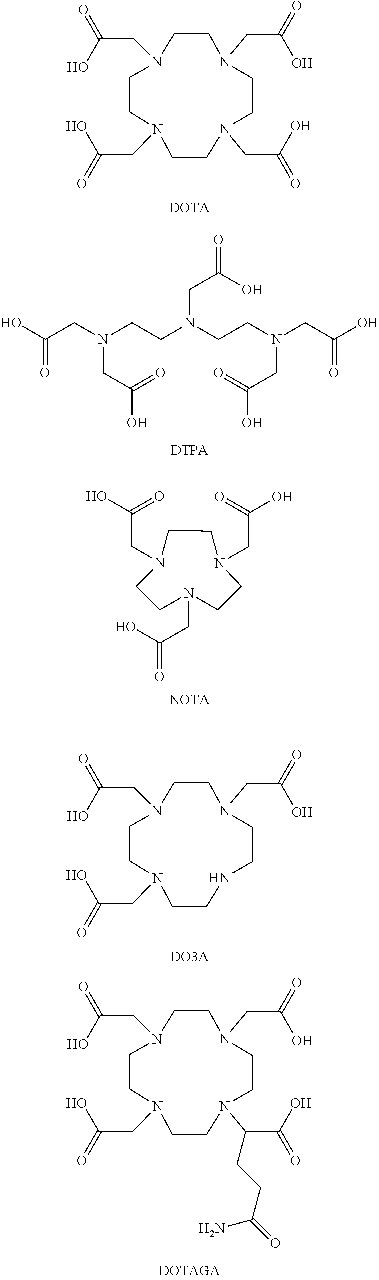

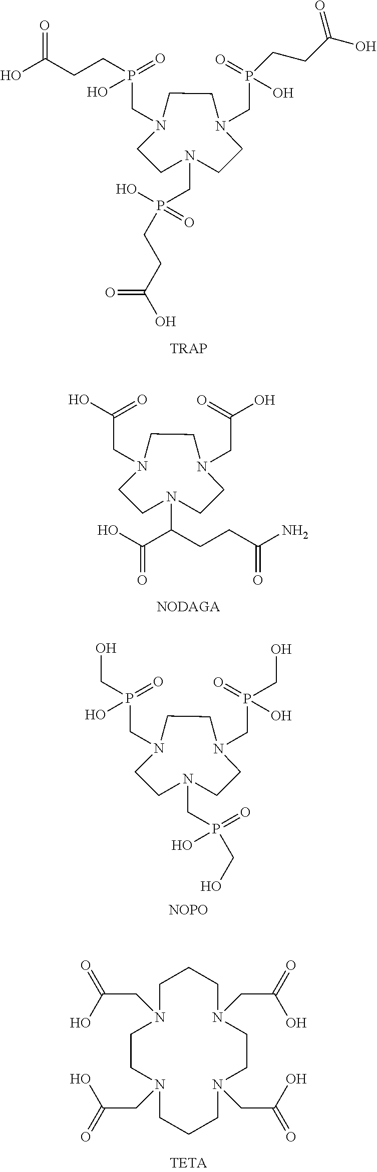 [SMILES: O=C(O)CCP(=O)(O)CN1CCN(CP(=O)(O)CCC(=O)O)CCN(CP(=O)(O)CCC(=O)O)CC1 ; NC(=O)CCC(C(=O)O)N1CCN(CC(=O)O)CCN(CC(=O)O)CC1 ; O=P(O)(CO)CN1CCN(CP(=O)(O)CO)CCN(CP(=O)(O)CO)CC1 ; "NOPO" ; O=C(O)CN1CCCN(CC(=O)O)CCN(CC(=O)O)CCCN(CC(=O)O)CC1]

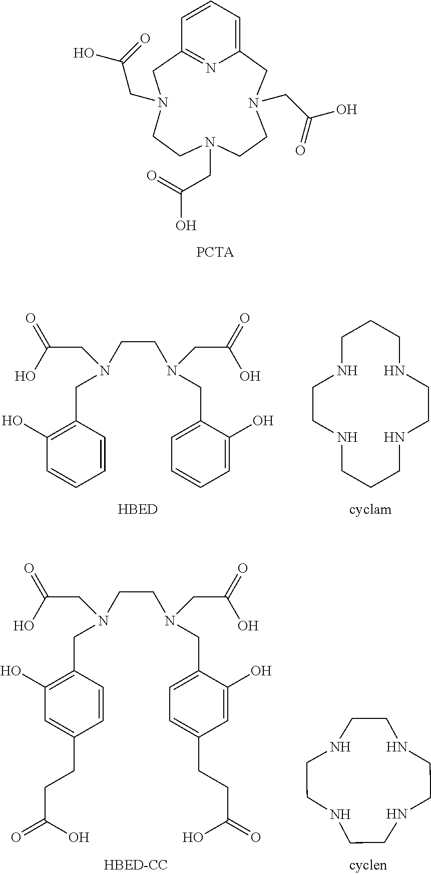 [SMILES: O=C(O)CN1CCN(CC(=O)O)Cc2cccc(n2)CN(CC(=O)O)CC1 ; O=C(O)CN(CCN(CC(=O)O)Cc1ccccc1O)Cc1ccccc1O ; C1CNCCNCCCNCCNC1 ; O=C(O)CCc1ccc(CN(CCN(CC(=O)O)Cc2ccc(CCC(=O)O)cc2O)CC(=O)O)c(O)c1 ; C1CNCCNCCNCCN1]

In certain embodiments, the linkers in the compound of Formula (I) are the same. In other embodiments, the linkers in the compound of Formula (I) are different. In certain embodiments, each linker comprises one or more of the following groups or fragments thereof:

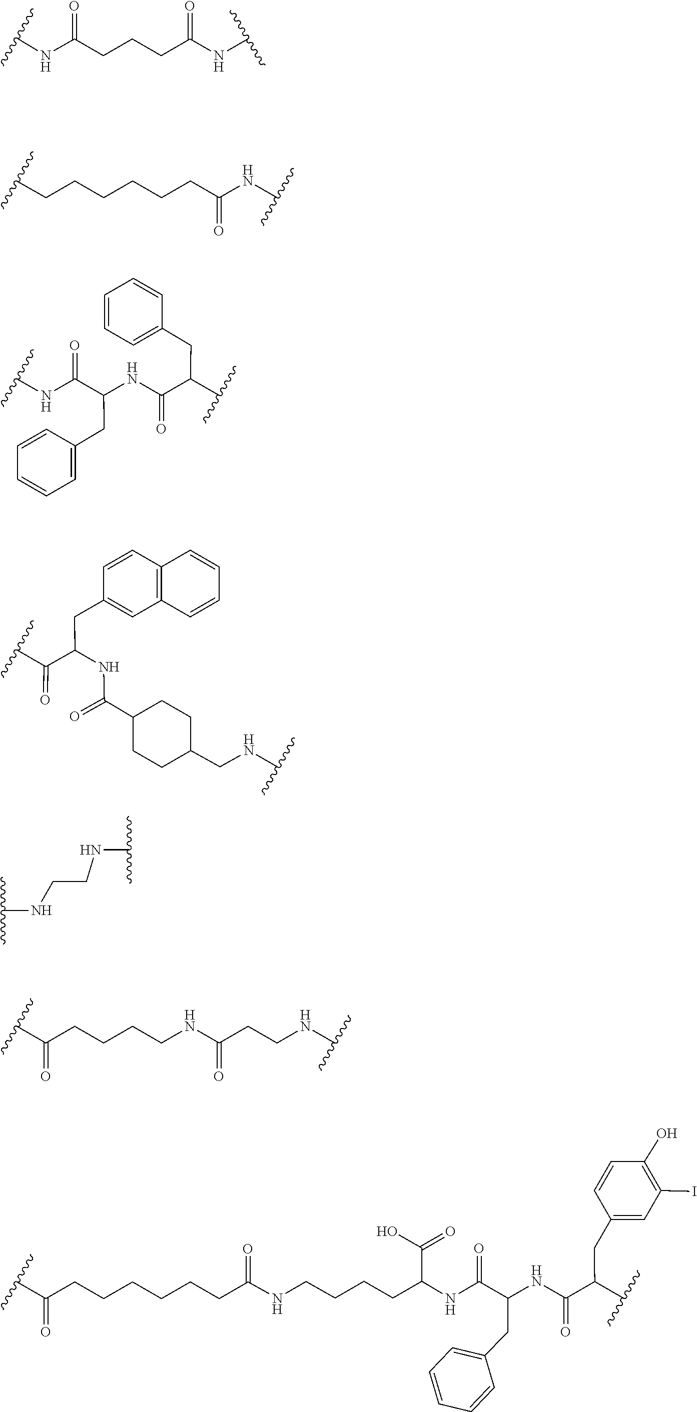

In some embodiments, the compound of Formula (I) has one of the following structures:

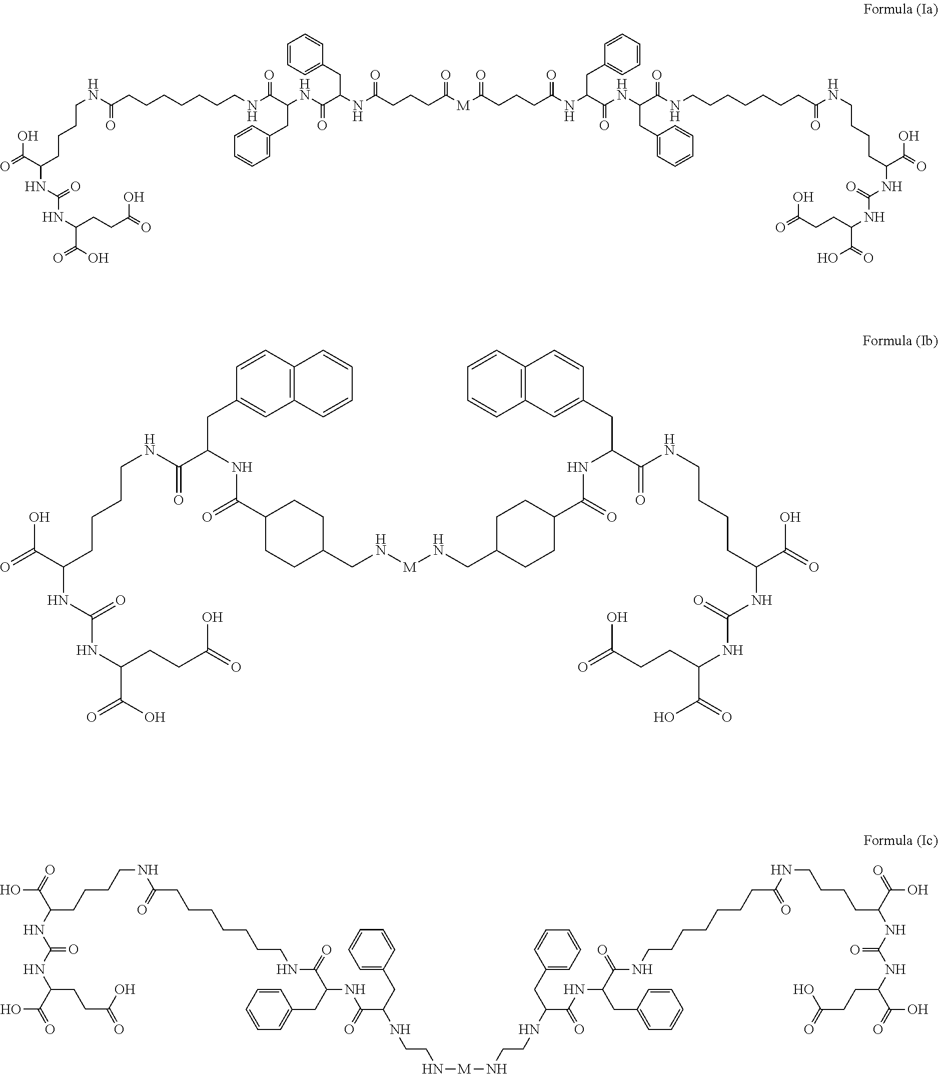

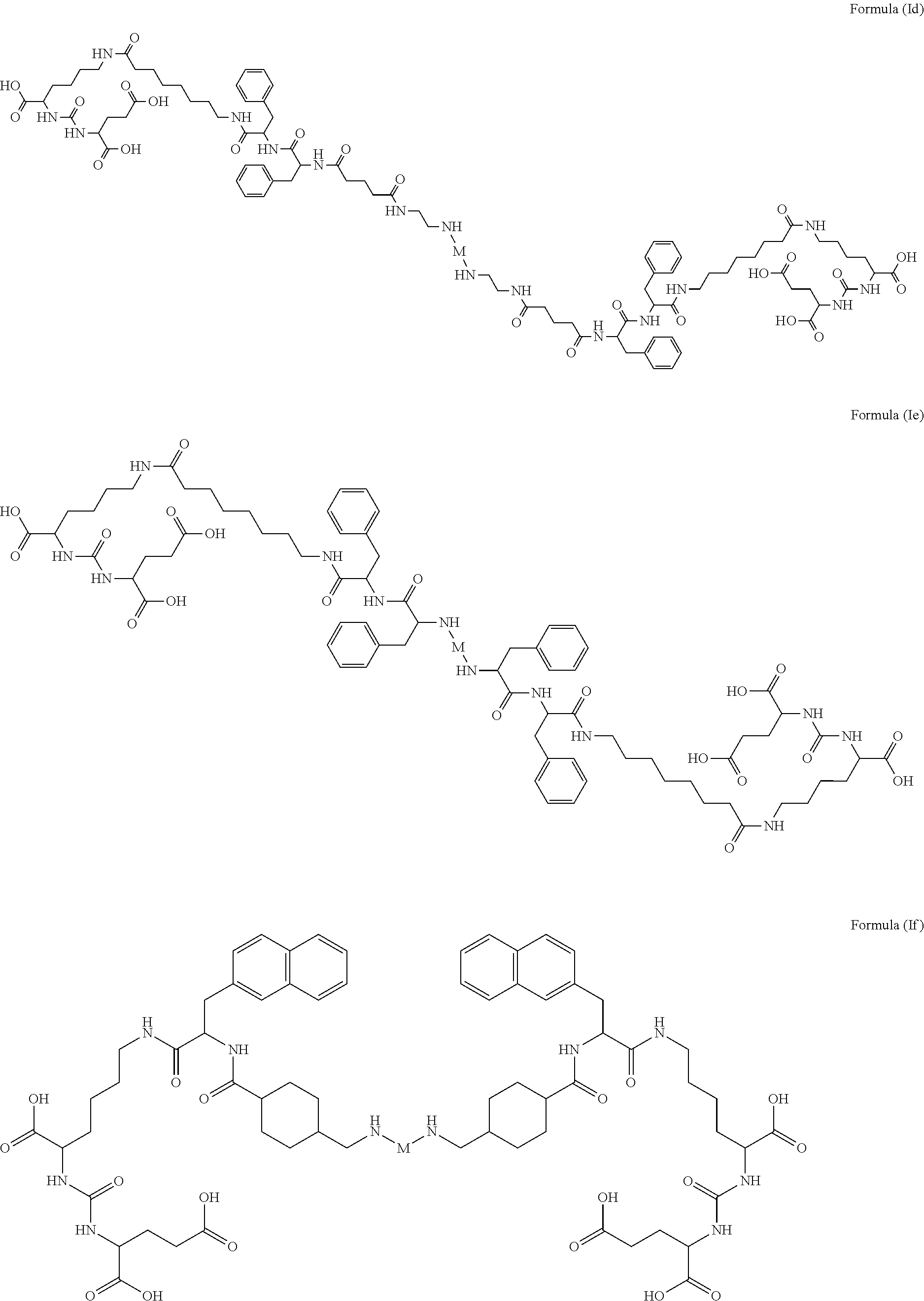

In certain embodiments, the metal chelator in the compound of Formula (I) is complexed with an ion of a metal selected from the group consisting of Cu, Lu, Ac, Tc, Gd, Ga, In, Co, Re, Fe, Mg, Ag, Rh, Pt, Cr, Ni, V, Ir, Zn, Cd, Mn, Ru, Pd, Hg, Ti, Lu, Sc, Zr, Y, Ac, As, Ra and Pb.

In some embodiments, the metal ion complexed in the metal chelator is a radionuclide.

In some embodiments, the compound of Formula (I) is complexed with a radionuclide selected from the group consisting of ⁶⁰Cu, ⁶¹Cu, ⁶²Cu, ⁶⁴Cu, ⁶⁷Cu, ⁶⁸Ga, ⁹⁰Y, ¹¹¹In, ¹⁷⁷Lu, ¹⁸⁸Re, ²¹¹As, ²¹²Pb and ²²⁵Ac.

In a second aspect, the present invention provides a composition comprising a compound of Formula (I) as defined in the first aspect and one or more pharmaceutically acceptable excipients.

The present inventors believe that the compounds coordinated with a radionuclide may be used as a radiopharmaceutical or radioimaging agent, if the compound can bind sufficiently to the desired site and deliver a radionuclide to the same site for the purposes of imaging or therapy.

In a third aspect, the present invention provides a method for radioimaging a cancer in a subject in need thereof, the method comprising administering to the subject a compound of Formula (I) according to the first aspect or a composition according to the second aspect, wherein the compound is coordinated with a radionuclide.

In certain embodiments, the method for radioimaging comprises imaging by positron emission tomography (PET) or single-photon emission computed tomography (SPECT).

In a fourth aspect, the present invention provides a method for the treatment of a cancer in a subject in need thereof, the method comprising administering to the subject a compound of Formula (I) according to the first aspect or a composition according to the second aspect, wherein the compound is coordinated with a radionuclide.

In particular embodiments, the cancer is characterised by overexpression of prostate-specific membrane antigen (PSMA). In further embodiments, the cancer is prostate cancer.

In a fifth aspect, the present invention provides a use of a compound of Formula (I) according to the first aspect in the manufacture of a medicament for radioimaging a cancer, wherein the compound is coordinated with a radionuclide.

In certain embodiments, the radioimaging comprises imaging by positron emission tomography (PET) or single-photon emission computed tomography (SPECT).

In a sixth aspect, the present invention provides a use of a compound of Formula (I) according to the first aspect in the manufacture of a medicament for treating a cancer, wherein the compound is coordinated with a radionuclide.

In certain embodiments, the cancer is characterised by the overexpression of prostate-specific membrane antigen (PSMA).

In other embodiments, the cancer is prostate cancer.

### BRIEF DESCRIPTION OF THE FIGURES

The invention will herein be described by way of example only with reference to the following non-limiting Figures in which:

FIG. 1 illustrates a graphical representation of the radio HPLC chromatograms for A) the HBED-bisPSMA precursor and B) the [⁶⁸Ga]Ga-HBED-bisPSMA sample.

FIG. 2 illustrates mean±SEM of injected dose (%) per gram of tissue weight (% ID/g) shown for each tissue for ⁶⁸Ga-HBED-CC-bis (PSMA), ⁶⁴Cu-DOTA-bis (PSMA) and ⁶⁴Cu-NOTA-bis (PSMA) at each of the two tissue biodistribution timepoints.

FIG. 3 illustrates mean±SEM of injected dose (%) per gram of tissue weight (% ID/g) shown for each tissue for ⁶⁸Ga-HBED-CC-bis (PSMA), ⁶⁴Cu-DOTA-bis (PSMA) and ⁶⁴Cu-NOTA-bis (PSMA) at each of the two tissue biodistribution timepoints with split y-axis.

FIG. 4 illustrates injected dose (%) per gram of tissue weight (% ID/g) shown for each tissue for ⁶⁸Ga-HBED-CC-bis (PSMA), ⁶⁴Cu-DOTA-bis (PSMA) and ⁶⁴Cu-NOTA-bis (PSMA) at each of the two tissue biodistribution timepoints, with data for individual mice shown (n=5 or 4) alongside mean±SEM

### DETAILED DESCRIPTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “about” or “approximately” as used herein means within an acceptable error range for the particular value as determined by one of ordinary skill in the art, which will depend in part on how the value is measured or determined, i.e., the limitations of the measurement system.

Unless defined otherwise, all technical and scientific terms used herein have the same meaning as commonly understood by those of ordinary skill in the art to which the invention belongs. For the purposes of the present invention, the following terms are defined below.

As used herein, the term “residue” refers to a part of a compound resulting from the removal of one or more atoms. The one or more atoms to be removed may be hydrogen atoms. A person skilled in the art would understand, for example, where a compound comprises a carboxylic acid (—COOH) functional group, the residue that is found in the compound of Formula (I) comprises the carboxylate of the amino acid (i.e. —COO⁻), which is attached to the remainder of the compound.

As used herein, the term “pharmaceutically acceptable salts” refers to salts that retain the desired biological activity of the above-identified compounds, and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic and arylsulfonic acids. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts may be prepared by reaction of a compound with the appropriate inorganic or organic acid via any of a number of known methods. Alternatively, alkali and alkaline earth metal salts can be prepared by reacting a compound with the appropriate base via a variety of known methods. The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates. Additional information on pharmaceutically acceptable salts can be found in Remington's Pharmaceutical Sciences, 19th Edition, Mack Publishing Co., Easton, PA 1995. In the case of agents that are solids, it is understood by those skilled in the art that the inventive compounds, agents and salts may exist in different crystalline or polymorphic forms, all of which are intended to be within the scope of the present invention and specified formulae.

As used herein, the term “complex” refers to a moiety comprising a ligand and a metal coordinated with a suitable part of the ligand. For example, the compounds of Formula (I) as disclosed herein acts as a ligand for one or more metal ions, where the metal ion is coordinated to the ligand via the metal chelator.

As used herein, used herein, the term “isomer” refers to and includes all regioisomers and stereoisomers of the compounds of the present invention. Examples of stereoisomers include diastereomers and enantiomers, where appropriate.

The compounds of Formula (I) disclosed herein contain a metal chelator (M). Examples of metal chelators (M) used in the compounds of the present invention include:

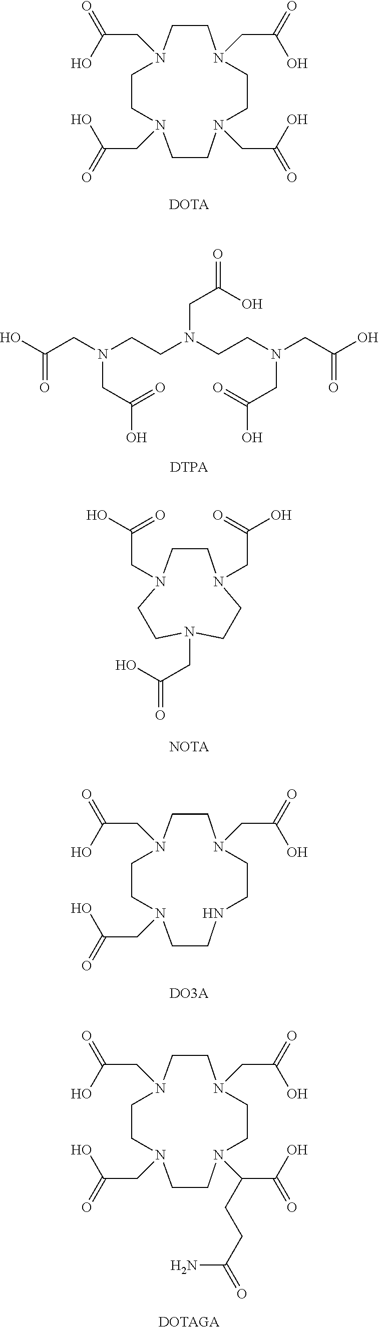

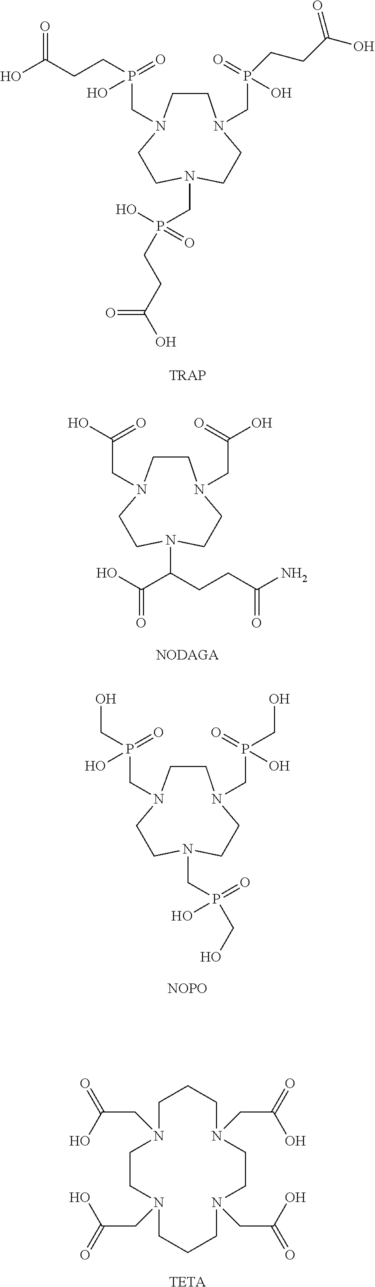

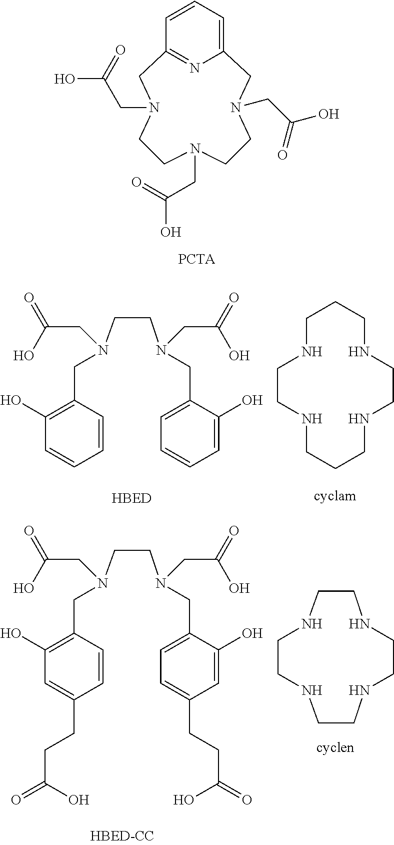

It will be understood that the residue of a metal chelator is selected to tightly bind the intended radionuclide for imaging or therapy and in light of the intended application for the compound. There are studies illustrating that conjugates comprising the same targeting compound but different macrocyclic chelators have different biodistribution profiles (von Witting, E. et al. European Journal of Pharmaceutics and Biopharmaceutics, 2019, 140, 109-120; Bogdan, M. et al. International Journal of Oncology, 2016, 2124-2134). Differences in biodistribution may be present even where the radionuclides have the same valency. Heppeler, A, et al. investigated a somatostatin analogue conjugated with DOTA and complexed with either ⁶⁷Ga or ⁹⁰Y (Heppeler, A, et al. Chemistry, 1999, 5, 1974-1981). The analogue complexed with ⁶⁷Ga had a 5-fold higher affinity to the somatostatin receptor type 2 and a 2-fold higher tumour uptake compared to the ⁹⁰Y complex. These pronounced differences were attributed to different coordination geometries of ⁶⁷Ga and ⁹⁰Y with DOTA, which resulted in conformational differences in the conjugated peptide.

In one embodiment, the present invention provides a compound of Formula (I):

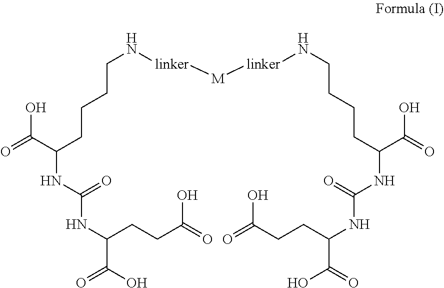

or a pharmaceutically acceptable salt, complex, isomer, solvate or prodrug thereof,
wherein each linker may be the same or different;
wherein M is a residue of a metal chelator selected from the group consisting of:

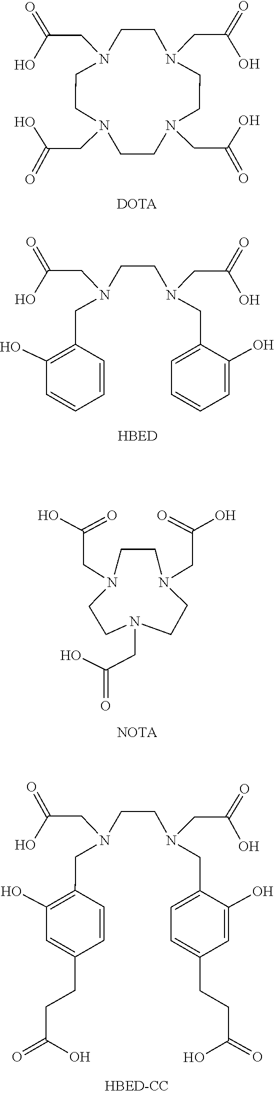

In another embodiment, the present invention provides a compound of Formula (I):

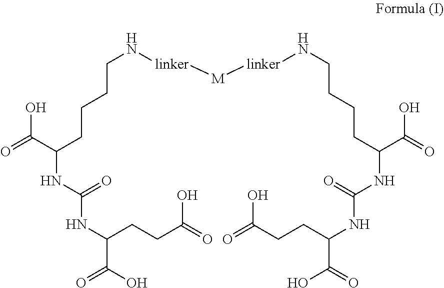

or a pharmaceutically acceptable salt, complex, isomer, solvate or prodrug thereof,
wherein each linker may be the same or different;
wherein M is a residue of a metal chelator selected from the group consisting of:

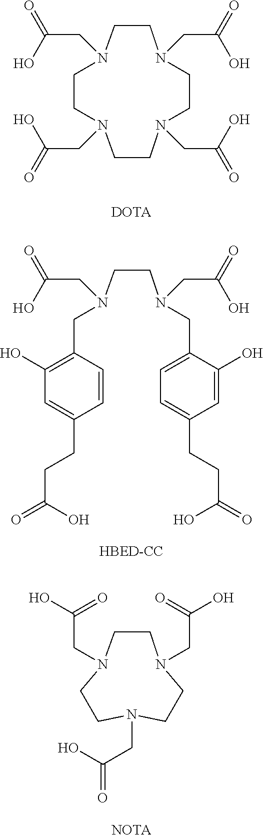

It will be appreciated that above embodiments of Formula (I) comprise chelators that are representative of macrocyclic chelators comprising three or four coordinating nitrogen atoms as well as acyclic chelators. The skilled addressee would reasonably expect embodiments of the invention comprising other macrocyclic or acyclic chelators to exhibit similar pharmacological properties to these embodiments.

Examples of compounds of the present invention containing a metal chelator include:

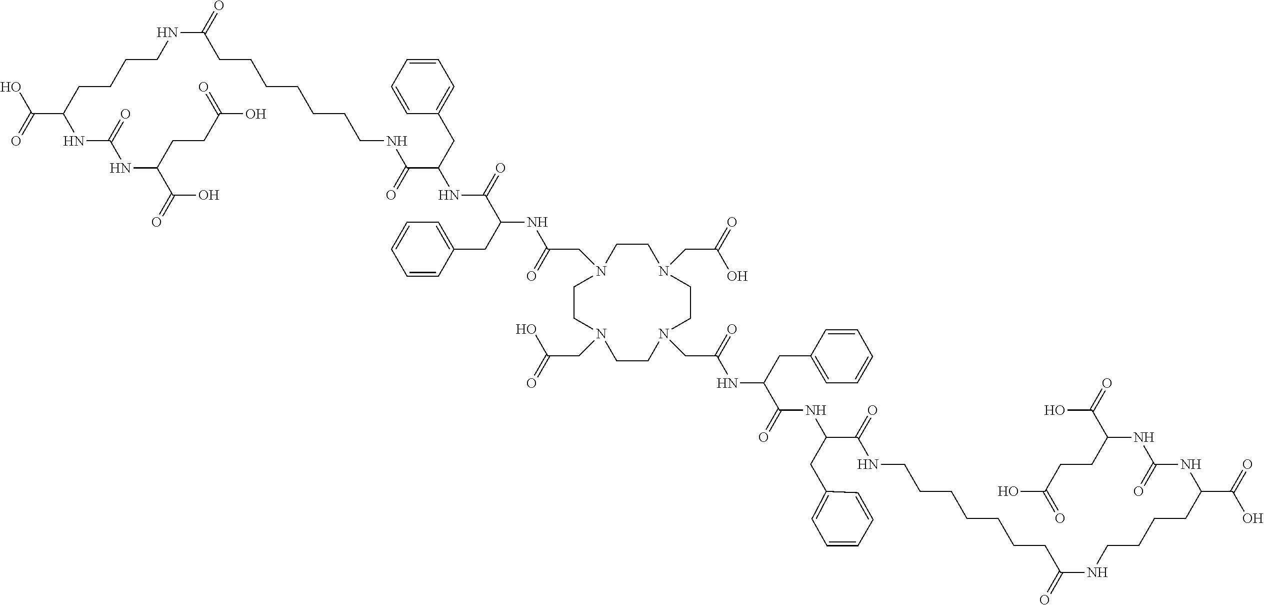 [SMILES: O=C(O)CCC(NC(=O)NC(CCCCNC(=O)CCCCCCCNC(=O)C(Cc1ccccc1)NC(=O)C(Cc1ccccc1)NC(=O)CN1CCN(CC(=O)O)CCN(CC(=O)NC(Cc2ccccc2)C(=O)NC(Cc2ccccc2)C(=O)NCCCCCCCC(=O)NCCCCC(NC(=O)NC(CCC(=O)O)C(=O)O)C(=O)O)CCN(CC(=O)O)CC1)C(=O)O)C(=O)O]

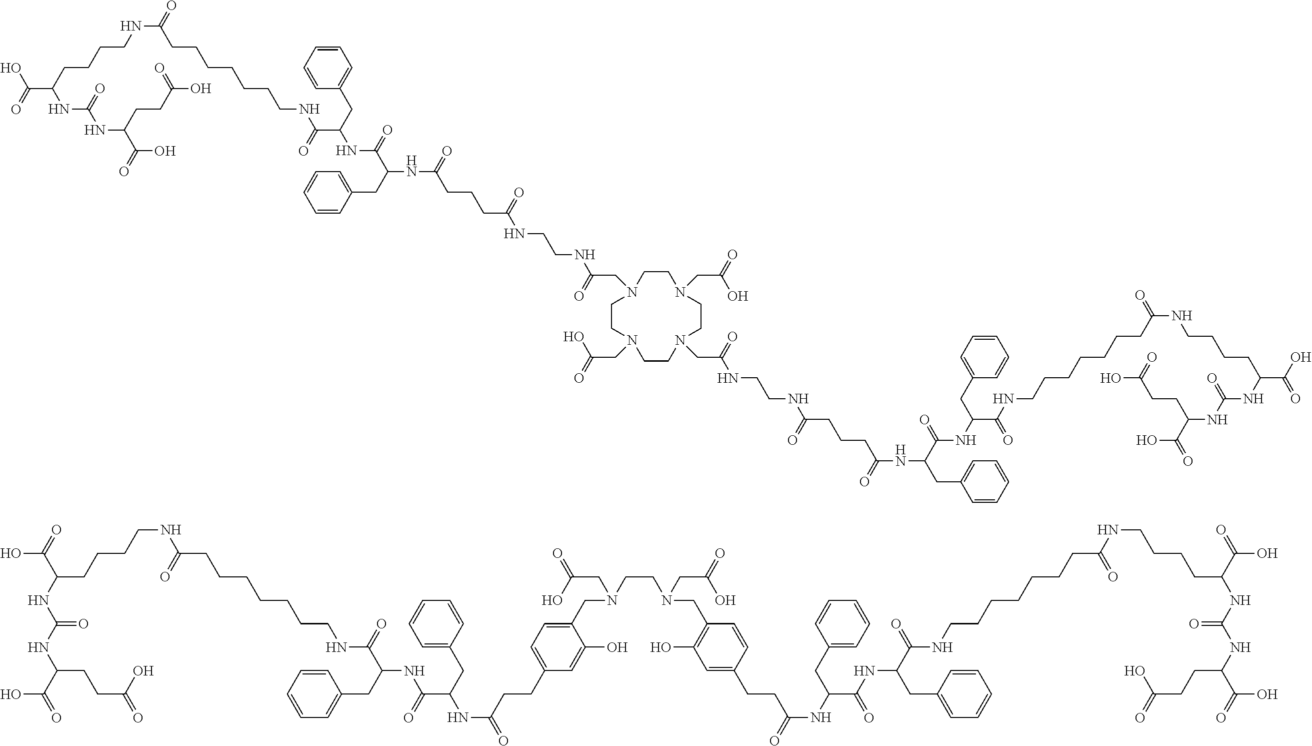 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NCCNC(=O)CN1CCN(CC(=O)O)CCN(CC(=O)NCCNC(=O)CCCC(=O)NC(Cc2ccccc2)C(=O)NC(Cc2ccccc2)C(=O)NCCCCCCCC(=O)NCCCCC(NC(=O)NC(CCC(=O)O)C(=O)O)C(=O)O)CCN(CC(=O)O)CC1)C(=O)O)C(=O)O ; O=C(O)CCC(NC(=O)NC(CCCCNC(=O)CCCCCCCNC(=O)C(Cc1ccccc1)NC(=O)C(Cc1ccccc1)NC(=O)CCc1ccc(CN(CCN(CC(=O)O)Cc2ccc(CCC(=O)NC(Cc3ccccc3)C(=O)NC(Cc3ccccc3)C(=O)NCCCCCCCC(=O)NCCCCC(NC(=O)NC(CCC(=O)O)C(=O)O)C(=O)O)cc2O)CC(=O)O)c(O)c1)C(=O)O)C(=O)O]

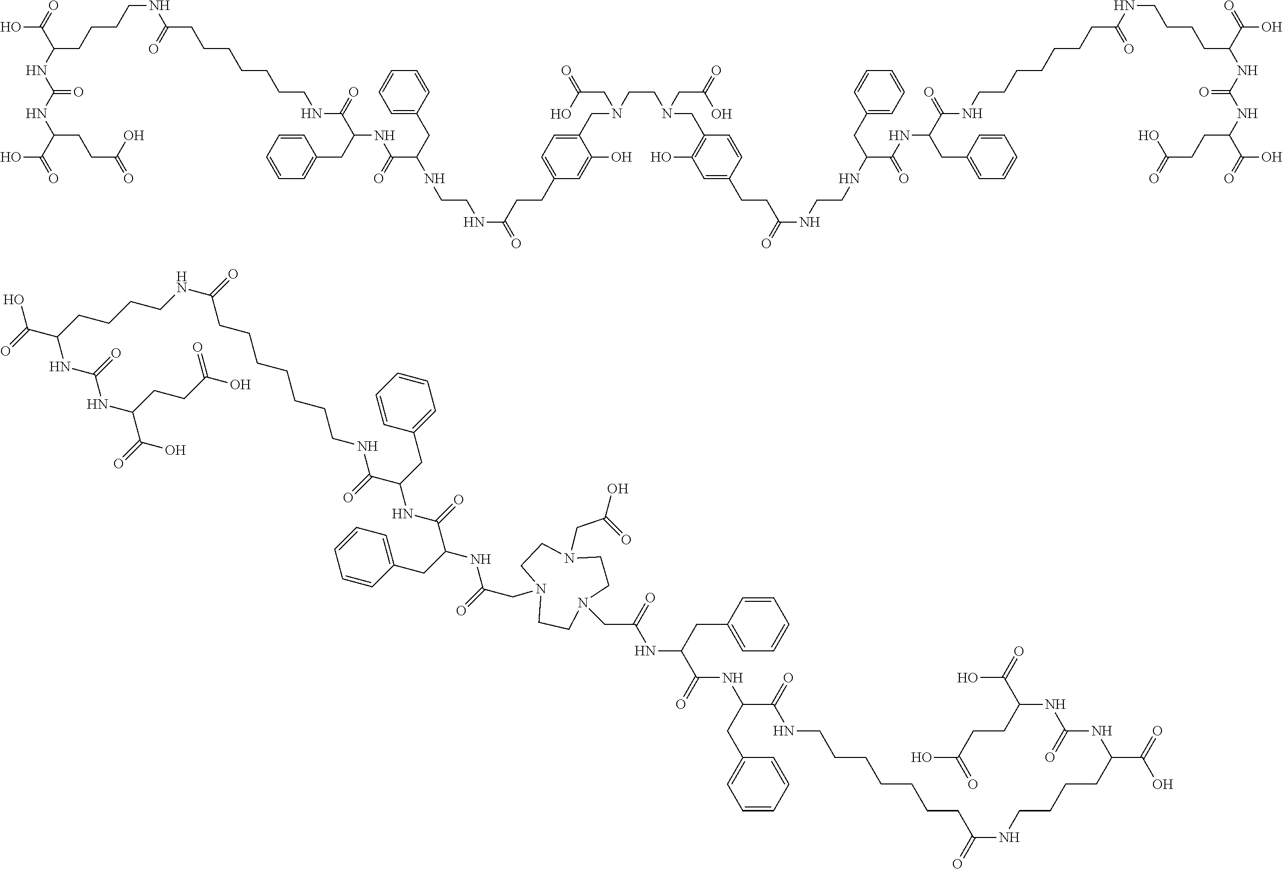 [SMILES: O=C(O)CCC(NC(=O)NC(CCCCNC(=O)CCCCCCCNC(=O)C(Cc1ccccc1)NC(=O)C(Cc1ccccc1)NCCNC(=O)CCc1ccc(CN(CCN(CC(=O)O)Cc2ccc(CCC(=O)NCCNC(Cc3ccccc3)C(=O)NC(Cc3ccccc3)C(=O)NCCCCCCCC(=O)NCCCCC(NC(=O)NC(CCC(=O)O)C(=O)O)C(=O)O)cc2O)CC(=O)O)c(O)c1)C(=O)O)C(=O)O ; O=C(O)CCC(NC(=O)NC(CCCCNC(=O)CCCCCCCNC(=O)C(Cc1ccccc1)NC(=O)C(Cc1ccccc1)NC(=O)CN1CCN(CC(=O)O)CCN(CC(=O)NC(Cc2ccccc2)C(=O)NC(Cc2ccccc2)C(=O)NCCCCCCCC(=O)NCCCCC(NC(=O)NC(CCC(=O)O)C(=O)O)C(=O)O)CC1)C(=O)O)C(=O)O]

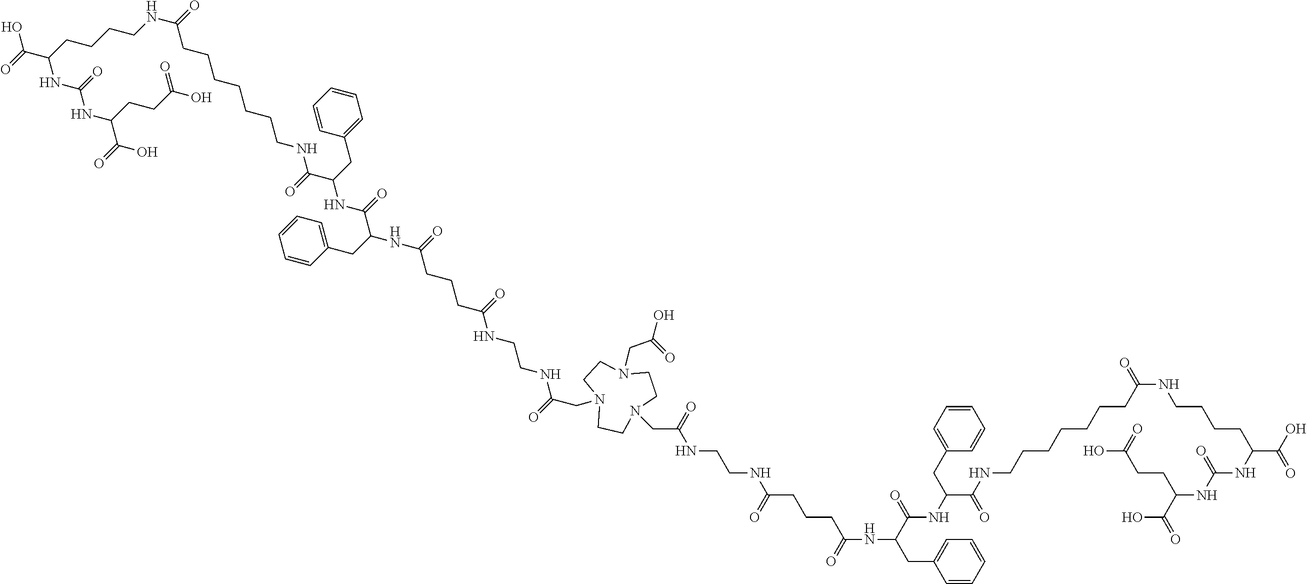 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NCCNC(=O)CN1CCN(CC(=O)O)CCN(CC(=O)NCCNC(=O)CCCC(=O)NC(Cc2ccccc2)C(=O)NC(Cc2ccccc2)C(=O)NCCCCCCCC(=O)NCCCCC(NC(=O)NC(CCC(=O)O)C(=O)O)C(=O)O)CC1)C(=O)O)C(=O)O]

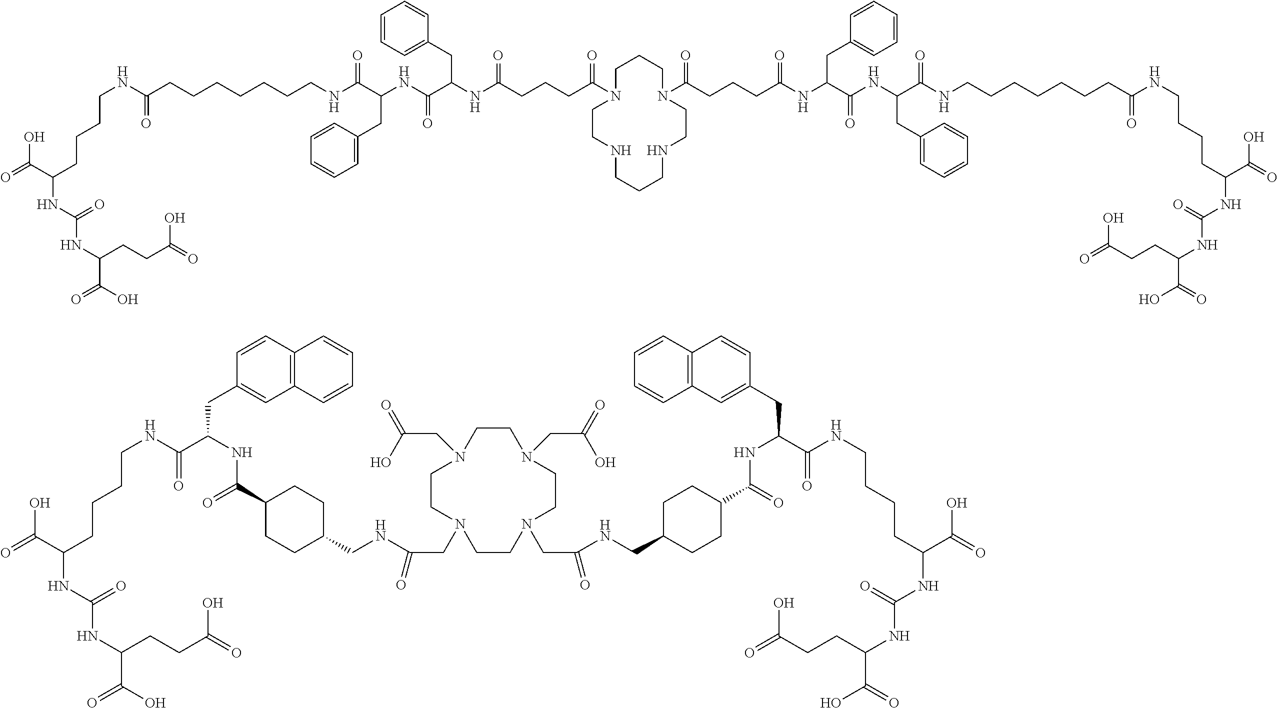

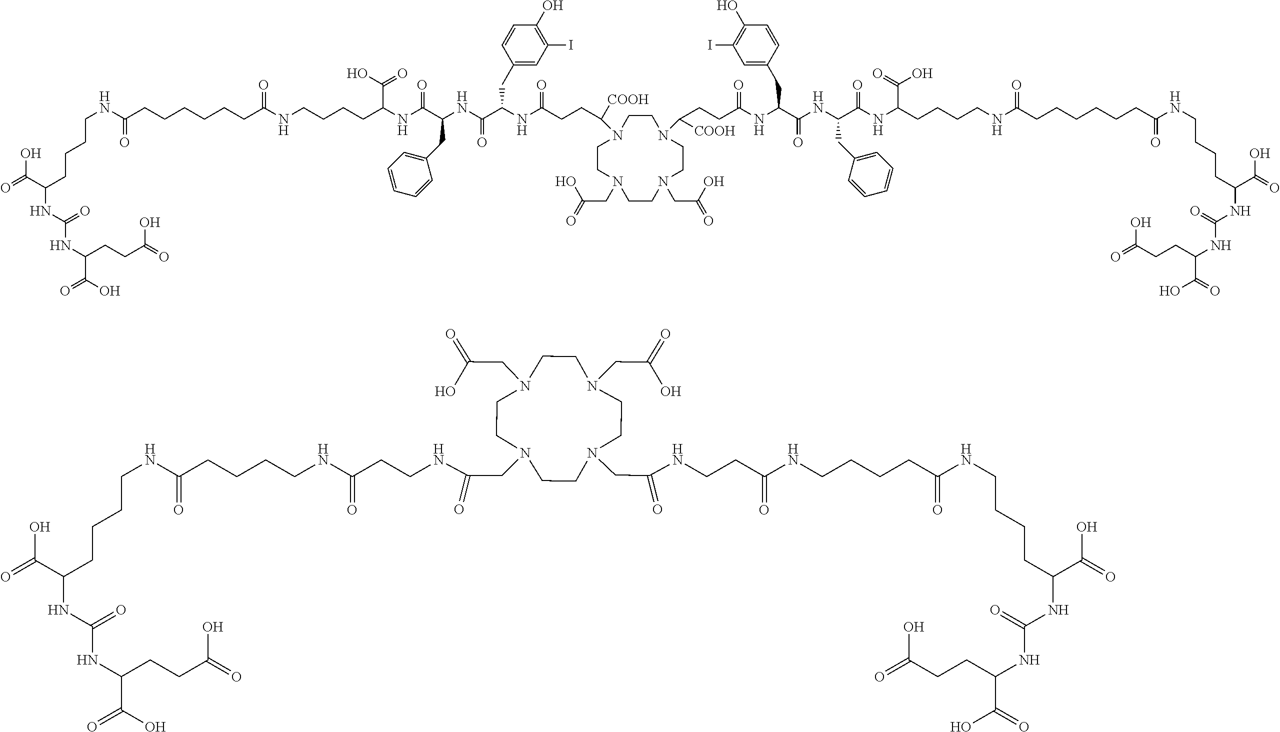 [SMILES: O=C(O)CCC(NC(=O)NC(CCCCNC(=O)CCCCCCC(=O)NCCCCC(NC(=O)[C@H](Cc1ccccc1)NC(=O)[C@H](Cc1ccc(O)c(I)c1)NC(=O)CCC(C(=O)O)N1CCN(CC(=O)O)CCN(CC(=O)O)CCN(C(CCC(=O)N[C@@H](Cc2ccc(O)c(I)c2)C(=O)N[C@@H](Cc2ccccc2)C(=O)NC(CCCCNC(=O)CCCCCCC(=O)NCCCCC(NC(=O)NC(CCC(=O)O)C(=O)O)C(=O)O)C(=O)O)C(=O)O)CC1)C(=O)O)C(=O)O)C(=O)O ; O=C(O)CCC(NC(=O)NC(CCCCNC(=O)CCCCNC(=O)CCNC(=O)CN1CCN(CC(=O)O)CCN(CC(=O)O)CCN(CC(=O)NCCC(=O)NCCCCC(=O)NCCCCC(NC(=O)NC(CCC(=O)O)C(=O)O)C(=O)O)CC1)C(=O)O)C(=O)O]

The structures depicted above represent a particular regioisomer of a compound of Formula (I). The present invention also contemplates different regioisomers of the compounds of the present invention, where the linkers may be attached to the metal chelator via different atoms of the chelator. Without wishing to be bound by theory, the present inventors believe that a particular regioisomer of a specific compound may provide advantages such as greater stability of the complex that is formed by complexation of the compound with a metal ion, optimal separation of the terminal groups attached to the linker and favourable shape and size of the compound overall. The structures depicted above also represent the racemic form (i.e. with undefined stereochemistry) of a particular compound or a particular stereoisomer of a particular compound. The present invention also contemplates different stereoisomers (including enantiomers and diastercomers) of the compounds described herein. Without wishing to be bound by theory, the present inventors believe that a specific enantiomeric form of a compound disclosed herein may provide greater advantages (such as increased binding affinity and overall efficacy) over its corresponding racemic form.

Examples of ions that may form complexes with the compounds of the present invention includes ions of a metal selected from the group consisting of Cu, Lu, Ac, Tc, Gd, Ga, In, Co, Rc, Fe, Mg, Ag, Rh, Pt, Cr, Ni, V, Ir, Zn, Cd, Mn, Ru, Pd, Hg, Ti, Lu, Sc, Zr, Lu, Sc. Zr, Y, Ac, As, Ra and Pb.

In some embodiments, the metal ion complexed in the metal chelator is a radionuclide.

In some embodiments, the compound of Formula (I) is complexed with a radionuclide selected from the group consisting of ⁶⁰Cu, ⁶¹Cu, ⁶²Cu, ⁶⁴Cu, ⁶⁷Cu, ⁶⁸Ga, ⁹⁰Y, ¹¹¹In, ¹⁷⁷Lu, ¹⁸⁸Re, ²¹¹As, ²¹²Pb and ²²⁵Ac.

The metal chelators described herein may form a complex with one or more metal ions where specific metal chelators may form a preferred complex with one or more metal ions. For example, compounds of the present invention containing DOTA as a metal chelator may form a complex with a Lu ion. In certain embodiments, a compound of the present invention includes a DOTA chelator and a Lu ion. In other embodiments, a compound of the present invention includes a DOTA chelator and a ¹⁷⁷Lu ion. In other embodiments, a compound of the present invention includes a DOTA chelator and an Ac ion. In some embodiments, a compound of the present invention includes a DOTA chelator and an ²²⁵Ac ion. In other embodiments, a compound of the present invention includes a DOTA chelator and an In ion. In some embodiments, a compound of the present invention includes a DOTA chelator and an ¹¹¹In ion. In other embodiments, a compound of the present invention includes a DOTA chelator and a Y ion. In some embodiments, a compound of the present invention includes a DOTA chelator and a ⁹⁰Y ion. In other embodiments, a compound of the present invention includes a DOTA chelator and a Re ion. In some embodiments, a compound of the present invention includes a DOTA chelator and a ¹⁸⁸Re ion. In other embodiments, a compound of the present invention includes a DOTA chelator and a Ga ion. In some embodiments, a compound of the present invention includes a DOTA chelator and a ⁶⁸Ga ion. In other embodiments, a compound of the present invention includes a DOTA chelator and a Cu ion. In some embodiments, a compound of the present invention includes a DOTA chelator and a ⁶⁷Cu ion. In other embodiments, a compound of the present invention includes a DOTA chelator and an As ion. In some embodiments, a compound of the present invention includes a DOTA chelator and a ²¹¹As ion.

In other embodiments, a compound of the present invention includes HBED-CC as a metal chelator that may form a complex with a Ga ion. In certain embodiments, a compound of the present invention includes a HBED-CC chelator and a Ga ion. In certain embodiments, a compound of the present invention includes a HBED-CC chelator and a ⁶⁸Ga ion. In other embodiments, a compound of the present invention includes HBED as a metal chelator that may form a complex with a Ga ion. In certain embodiments, a compound of the present invention includes a HBED chelator and a Ga ion. In certain embodiments, a compound of the present invention includes a HBED chelator and a ⁶⁸Ga ion.

In other embodiments, a compound of the present invention includes a NOTA chelator and an Ac ion. In some embodiments, a compound of the present invention includes a NOTA chelator and an ²²⁵Ac ion. In other embodiments, a compound of the present invention includes a NOTA chelator and an In ion. In some embodiments, a compound of the present invention includes a NOTA chelator and an ¹¹¹In ion. In other embodiments, a compound of the present invention includes a NOTA chelator and a Y ion. In some embodiments, a compound of the present invention includes a NOTA chelator and a ⁹⁰Y ion. In other embodiments, a compound of the present invention includes a NOTA chelator and a Re ion. In some embodiments, a compound of the present invention includes a NOTA chelator and a ¹⁸⁸Re ion. In other embodiments, a compound of the present invention includes a NOTA chelator and a Ga ion. In some embodiments, a compound of the present invention includes a NOTA chelator and a ⁶⁸Ga ion. In other embodiments, a compound of the present invention includes a NOTA chelator and a Cu ion. In some embodiments, a compound of the present invention includes a NOTA chelator and a ⁶⁷Cu ion.

In other embodiments, a compound of the present invention includes a DOTAGA chelator and an Ac ion. In some embodiments, a compound of the present invention includes a DOTAGA chelator and an ²²⁵Ac ion. In other embodiments, a compound of the present invention includes a DOTAGA chelator and an In ion. In some embodiments, a compound of the present invention includes a DOTAGA chelator and an ¹¹¹In ion. In other embodiments, a compound of the present invention includes a DOTAGA chelator and a Y ion. In some embodiments, a compound of the present invention includes a DOTAGA chelator and a ⁹⁰Y ion. In other embodiments, a compound of the present invention includes a DOTAGA chelator and a Re ion. In some embodiments, a compound of the present invention includes a DOTAGA chelator and a ¹⁸⁸Re ion. In other embodiments, a compound of the present invention includes a DOTAGA chelator and a Ga ion. In some embodiments, a compound of the present invention includes a DOTAGA chelator and a ⁶⁸Ga ion. In other embodiments, a compound of the present invention includes a DOTAGA chelator and a Cu ion. In some embodiments. In some embodiments, a compound of the present invention includes a DOTAGA chelator and a ⁶⁷Cu ion.

In other embodiments, a compound of the present invention includes a DOTAGA chelator and an Ac ion. In some embodiments, a compound of the present invention includes a DOTAGA chelator and an ²²⁵Ac ion. In other embodiments, a compound of the present invention includes a NOTAGA chelator and an In ion. In some embodiments, a compound of the present invention includes a NOTAGA chelator and an ¹¹¹In ion. In other embodiments, a compound of the present invention includes a NOTAGA chelator and a Y ion. In some embodiments, a compound of the present invention includes a NOTAGA chelator and a ⁹⁰Y ion. In other embodiments, a compound of the present invention includes a NOTAGA chelator and a Re ion. In some embodiments, a compound of the present invention includes a NOTAGA chelator and a ¹⁸⁸Re ion. In other embodiments, a compound of the present invention includes a NOTAGA chelator and a Ga ion. In some embodiments, a compound of the present invention includes a NOTAGA chelator and a ⁶⁸Ga ion. In other embodiments, a compound of the present invention includes a NOTAGA chelator and a Cu ion. In some embodiments. In some embodiments, a compound of the present invention includes a NOTAGA chelator and a ⁶⁷Cu ion.

As used herein, the term “solvate” refers to a complex of the compound, where the complex may have variable stoichiometry formed by a solute and a solvent. Such solvents in the solvate should not interfere with the biological activity of the solute. Examples of suitable solvents may include water, ethanol or acetic acid. Methods of solvation of the compound are generally known in the art.

As used herein, the term “prodrug” refers to and includes derivatives that are converted in vivo to the compounds of the present invention. Such derivatives would readily occur to those skilled in the art, and include, for example, compounds containing a free hydroxyl group that is converted into an ester derivative, or containing a ring nitrogen atom that is converted to an N-oxide. Examples of ester derivatives include alkyl esters, phosphate esters and those formed from amino acids.

As used herein the terms “treating”, “treatment”, “preventing”, “prevention” and grammatical equivalents refer to any and all uses which remedy the stated neuroendocrine tumour, prevent, retard or delay the establishment of the disease, or otherwise prevent, hinder, retard, or reverse the progression of the disease. Thus the terms “treating” and “preventing” and the like are to be considered in their broadest context. For example, treatment does not necessarily imply that a patient is treated until total recovery. Where the disease displays or a characterized by multiple symptoms, the treatment or prevention need not necessarily remedy, prevent, hinder, retard, or reverse all of said symptoms, but may prevent, hinder, retard, or reverse one or more of said symptoms.

As used herein, the term “cancer” broadly encompasses neoplastic diseases characterised by abnormal cell growth with the potential to invade or spread to other parts of the body. The cancer may be benign, which does not spread to other parts of the body. The cancer may be malignant, meaning that the cancer cells can spread through the circulatory system or lymphatic system. The term as used herein includes all malignant, i.e. cancerous, disease states. The cancer may be present as a tumour.

As used herein, the term “cancer” broadly encompasses neoplastic diseases characterised by abnormal cell growth with the potential to invade or spread to other parts of the body. The cancer may be benign, which does not spread to other parts of the body. The cancer may be malignant, meaning that the cancer cells can spread through the circulatory system or lymphatic system. The term as used herein includes all malignant, i.e. cancerous, disease states. The cancer may be present as a tumour. Accordingly, the term “tumour” is used generally to define any malignant cancerous or pre-cancerous cell growth, and may include leukemias, but is particularly directed to solid tumours or carcinomas such as melanomas, colon, lung, ovarian, skin, breast, pancreas, pharynx, brain, prostate, liver, CNS, and renal cancers (as well as other cancers).

In certain embodiments, the cancer is associated with overexpression of prostate-specific membrane antigen (PSMA). In specific embodiments, the cancer is selected from the group consisting of breast cancer, colon cancer, lung cancer, ovarian cancer, prostate cancer, head and/or neck cancer, or renal, gastric, pancreatic cancer, hepatocellular carcinoma (HCC), brain cancer and a hematologic malignancy such as lymphoma or leukaemia. In particular embodiments, the cancer is prostate cancer. In other embodiments, the prostate cancer is metastatic castration-resistant prostate cancer.

The present invention contemplates the use of the compounds of Formula (I) complexed with a suitable radionuclide for the treatment of a cancer in a subject. In some embodiments, a compound of Formula (I) complexed with a radionuclide may be used for the treatment of a cancer that is associated with overexpression of PSMA. In other embodiments, a compound of the present invention complexed with a radionuclide is used for the treatment of a cancer selected from the group consisting of breast cancer, colon cancer, lung cancer, ovarian cancer, prostate cancer, head and/or neck cancer, or renal, gastric, pancreatic cancer, hepatocellular carcinoma (HCC), brain cancer and a hematologic malignancy such as lymphoma or leukaemia. In specific embodiments, a compound of the present invention complexed with a radionuclide is used for the treatment of a prostate cancer.

The present invention also discloses the use of the compounds of Formula (I) complexed with a suitable radionuclide for radioimaging a subject. In certain embodiments, the compounds of Formula (I) complexed with a suitable radionuclide is used for radioimaging a cancer that is associated with overexpression of PSMA. In other embodiments, a compound of the present invention is used for the radioimaging of a cancer selected from the group consisting of breast cancer, colon cancer, lung cancer, ovarian cancer, prostate cancer, head and/or neck cancer, or renal, gastric, liver, pancreatic cancer, brain cancer and a hematologic malignancy such as lymphoma or leukaemia. In other embodiments, a compound of Formula (I) complexed with a radionuclide is used for the radioimaging of a prostate cancer.

The term “subject” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. Preferably, the mammal is human or a laboratory test animal. Even more preferably, the mammal is a human.

The term “therapeutically effective amount” or “effective amount” is an amount sufficient to effect beneficial or desired clinical results. An effective amount can be administered in one or more administrations. For the purposes of radioimaging, an effective amount is sufficient for an image showing the localisation of the compound of Formula (I) administered to the subject, owing to the detection of the products of decay from the radioisotope that is complexed with the compound. For the purposes of treatment, an effective amount is typically sufficient to palliate, ameliorate, stabilize, reverse, slow and/or delay the progression of the cancer.

The compounds are typically used in the form of pharmaceutical compositions that are formulated depending on the desired mode of administration. The compositions are prepared in manners well known in the art.

In the above embodiments, the compositions of the present invention comprise ethanol as a component. The ethanol used in the composition may be anhydrous ethanol. Alternatively, the ethanol used in the composition may not have been subject to drying processes and may be hydrated. The ethanol is preferably pharmaceutical grade ethanol. The ethanol present in the composition may assist in preventing radiolysis of the radiolabelled complex of Formula (I).

In the above embodiments, the compositions of the present invention also comprise sodium chloride as a component. The sodium chloride in the formulations of the present invention may be provided as a saline solution. A saline solution is defined as an aqueous solution of sodium chloride. For example, normal saline is defined as an aqueous solution of sodium chloride at a concentration of 0.9% (w/v). In an embodiment of the present invention, the sodium chloride of a formulation is provided by a saline solution.

In the above embodiments, the compositions of the present invention comprise gentisic acid, or pharmaceutically acceptable salts and/or hydrates thereof, as a component. Gentisic acid is also known as 2,5-dihydroxybenzoic acid, 5-hydroxysalicylic acid or hydroquinonecarboxylic acid. Salts of gentisic acid may include the sodium salt and the sodium salt hydrate. Any reference to gentisic acid may include a reference to salts thereof, where relevant. It has been identified by the present inventors that the gentisic acid or salt thereof, within the present composition may assist in preventing or minimising radiolysis of the radiolabelled complex of Formula (I).

The invention in other embodiments provides a pharmaceutical pack or kit comprising one or more containers filled with one or more of the ingredients of the pharmaceutical compositions of the invention. In such a pack or kit can be found at least one container having a unit dosage of the agent(s). Conveniently, in the kits, single dosages can be provided in sterile vials so that the clinician can employ the vials directly, where the vials will have the desired amount and concentration of compound and radionuclide, which may be admixed prior to use. Associated with such container(s) can be various written materials such as instructions for use, or a notice in the form prescribed by a governmental agency regulating the manufacture, use or sale of pharmaceuticals, imaging agents or biological products, which notice reflects approval by the agency of manufacture, use or sale for human administration.

The compounds of the invention may be used or administered in combination with one or more additional drug(s) that are anti-cancer drugs and/or procedures (e.g. surgery, radiotherapy) for the treatment of the disorder/diseases mentioned. The components can be administered in the same formulation or in separate formulations. If administered in separate formulations the compounds of the invention may be administered sequentially or simultaneously with the other drug(s).

In addition to being able to be administered in combination with one or more additional drugs that include anti-cancer drugs, the compounds of the invention may be used in a combination therapy. When this is done, the compounds are typically administered in combination with each other. Thus one or more of the compounds of the invention may be administered either simultaneously (as a combined preparation) or sequentially in order to achieve a desired effect. This is especially desirable where the therapeutic profile of each compound is different such that the combined effect of the two drugs provides an improved therapeutic result.

Pharmaceutical compositions of this invention for parenteral injection comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin.

If desired, and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions that can be dissolved or dispersed in sterile water or other sterile injectable medium just prior to use.

In another aspect, the present invention also provides a process for the preparation of compounds of Formula (I) or a pharmaceutically acceptable salt thereof as defined herein.

The compounds of Formula (I) may be prepared by a series of peptide coupling steps using appropriate amine and carboxylic acid derivatives. The coupling partners may require the installation of one or more protecting groups, which are subsequently removed after the coupling reaction. A list of suitable protecting groups in organic synthesis and procedures for their installation and removal can be found in T. W. Greene's Protective Groups in Organic Synthesis, 3rd Edition, John Wiley & Sons, 1991. Where a peptide coupling step is used to prepare the compounds of the present invention or a precursor thereof, the reaction may be performed under solution phase or solid phase conditions in the presence of one or more bases or other reagents as required. The coupling partners used in a given reaction step may be modified with one or more suitable groups that will facilitate the coupling reaction, for example, a leaving group. The preparation of compounds of Formula (I) may include the selection and installation of one or more protecting groups or the like that facilitate the coupling of components with the required site selectivity.

For example, the steps required to prepare a compound of Formula (I) may include the installation of one or more nitrogen or oxygen protecting groups installed at one or more amine or carboxylic acid functional groups.

As used herein, the term “oxygen protecting group” refers to a group that can prevent the oxygen moiety reacting during further derivatisation of the protected compound and which can be readily removed when desired. In one embodiment, the protecting group is removable in the physiological state by natural metabolic processes. Examples of oxygen protecting groups include acyl groups (such as acetyl), ethers (such as methoxy methyl ether (MOM), α-methoxy ethoxy methyl ether (MEM), p-methoxy benzyl ether (PMB), methylthio methyl ether, pivaloyl (Piv), tetrahydropyran (THP)), and silyl ethers (such as trimethylsilyl (TMS) tert-butyl dimethyl silyl (TBDMS) and triisopropylsilyl (TIPS) groups.

As used herein, the term “nitrogen protecting group” refers to a group that can prevent the nitrogen moiety reacting during further derivatisation of the protected compound and which can be readily removed when desired. In one embodiment, the protecting group is removable in the physiological state by natural metabolic processes and in essence the protected compound is acting as a prodrug for the active unprotected species. Examples of suitable nitrogen protecting groups that may be used include formyl, trityl, phthalimido, acetyl, trichloroacetyl, chloroacetyl, bromoacetyl, iodoacetyl; urethane-type blocking groups such as benzyloxycarbonyl (CBz), 4-phenylbenzyloxycarbonyl, 2-methylbenzyloxycarbonyl, 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, t-butoxycarbonyl (tBoc), 2-(4-xenyl)-isopropoxycarbonyl, 1,1-diphenyleth-1-yloxycarbonyl, 1,1-diphenylprop-1-yloxycarbonyl, 2-phenylprop-2-yloxycarbonyl, 2-(p-toluyl)-prop-2-yloxy-carbonyl, cyclo-pentanyloxy-carbonyl, 1-methylcyclopentanyloxycarbonyl, cyclohexanyloxycarbonyl, 1-methylcyclohexanyloxycarbonyl, 2-methylcyclohexanyloxycarbonyl, 2-(4-toluylsulfono)-ethoxycarbonyl, 2-(methylsulfono) ethoxycarbonyl, 2-(triphenylphosphino)-ethoxycarbonyl, fluorenylmethoxycarbonyl (Fmoc), 2-(trimethylsilyl) ethoxycarbonyl, allyloxycarbonyl, 1-(trimethylsilylmethyl) prop-1-enyloxycarbonyl, 5-benzisoxalylmethoxy carbonyl, 4-acetoxybenzyloxycarbonyl, 2,2,2-trichloroethoxycarbonyl, 2-ethynyl-2-propoxycarbonyl, cyclopropylmethoxycarbonyl, 4-(decyloxy)benzyloxycarbonyl, isobornyloxycarbonyl, 1-piperidyloxycarbonlyl and the like; benzoylmethylsulfono group, 2-nitrophenylsulfenyl, diphenylphosphine oxide, and the like. The actual nitrogen protecting group employed is not critical so long as the derivatised nitrogen group is stable to the condition of subsequent reaction(s) and can be selectively removed as required without substantially disrupting the remainder of the molecule including any other nitrogen protecting group(s). Further examples of these groups are found in: Greene, T. W. and Wuts, P. G. M., Protective Groups in Organic Synthesis, Second edition; Wiley-Interscience: 1991; Chapter 7; McOmie, J. F. W. (ed.), Protective Groups in Organic Chemistry, Plenum Press, 1973; and Kocienski, P. J., Protecting Groups, Second Edition, Thieme Medical Pub., 2000.

The compounds of Formula (I) or a pharmaceutically acceptable salt thereof as disclosed herein may be synthesised according to Scheme 1:

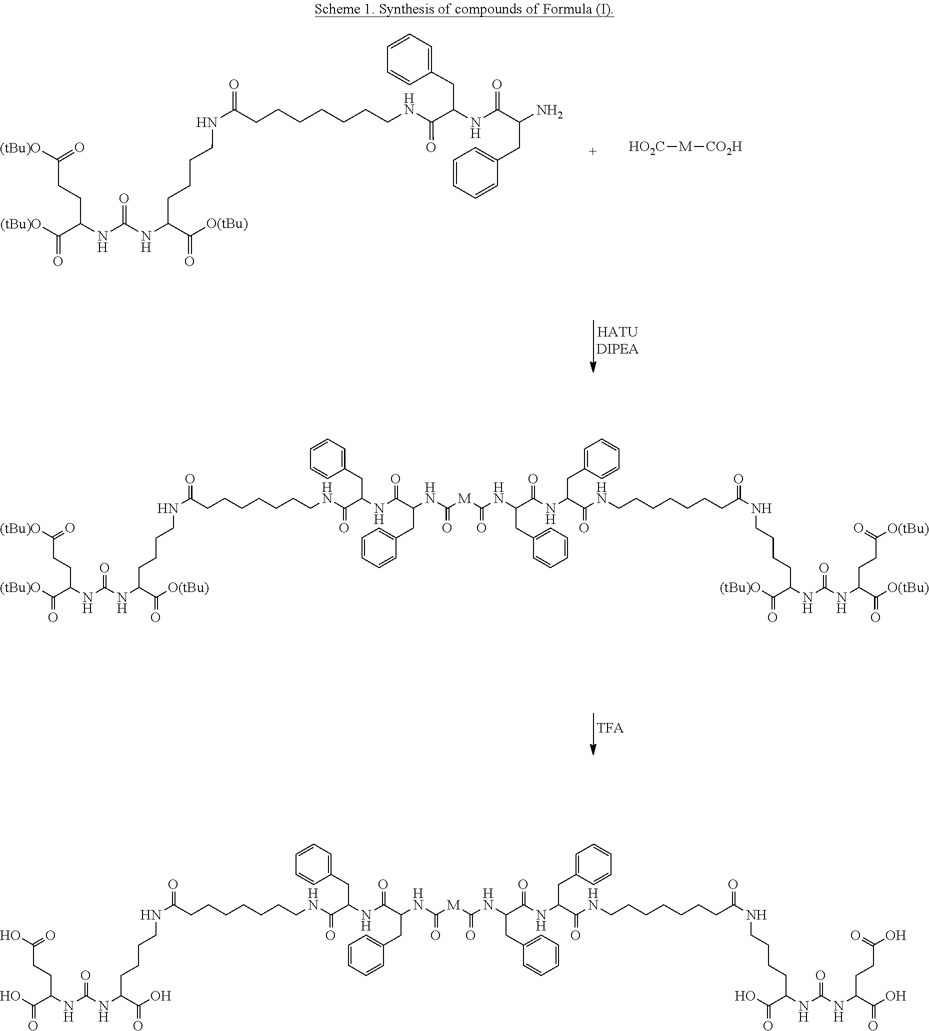

Scheme 1 describes a synthesis of compounds of Formula (I) where the metal chelator (M) is coupled with two moieties of the linker-PSMA urea group at the same time. The reaction may be performed under standard peptide coupling conditions with a suitable peptide coupling reagent and a base, where the linker-PSMA urea group contains the amine functional group and the metal chelator contains the carboxylic acid functional group that will participate in the coupling reaction.

Alternatively, the compounds of Formula (I) or a pharmaceutically acceptable salt thereof as disclosed herein may be synthesised according to Scheme 2:

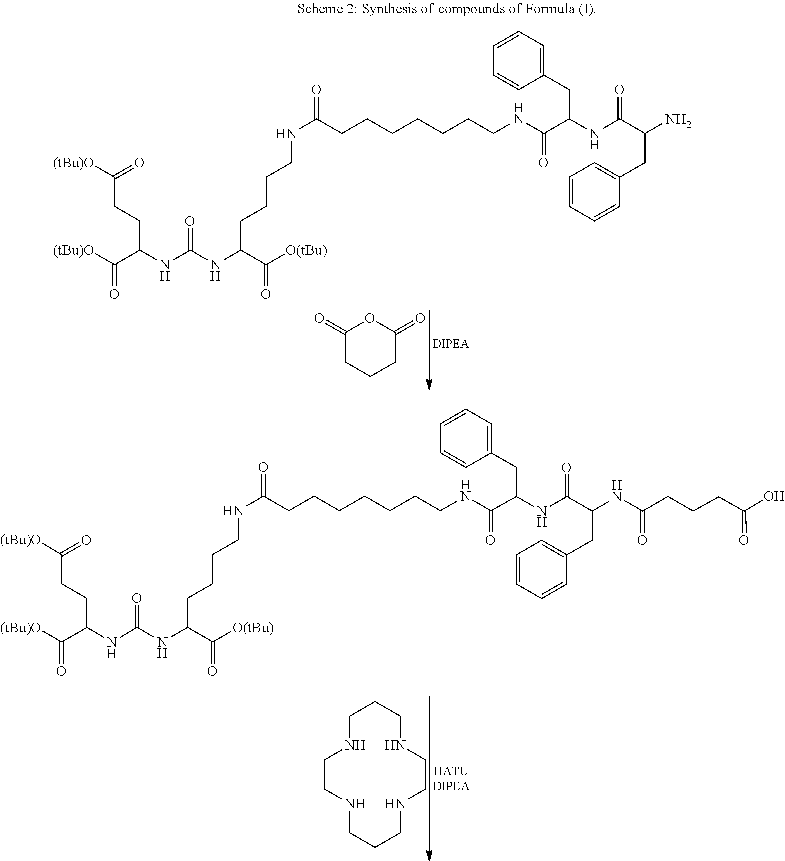

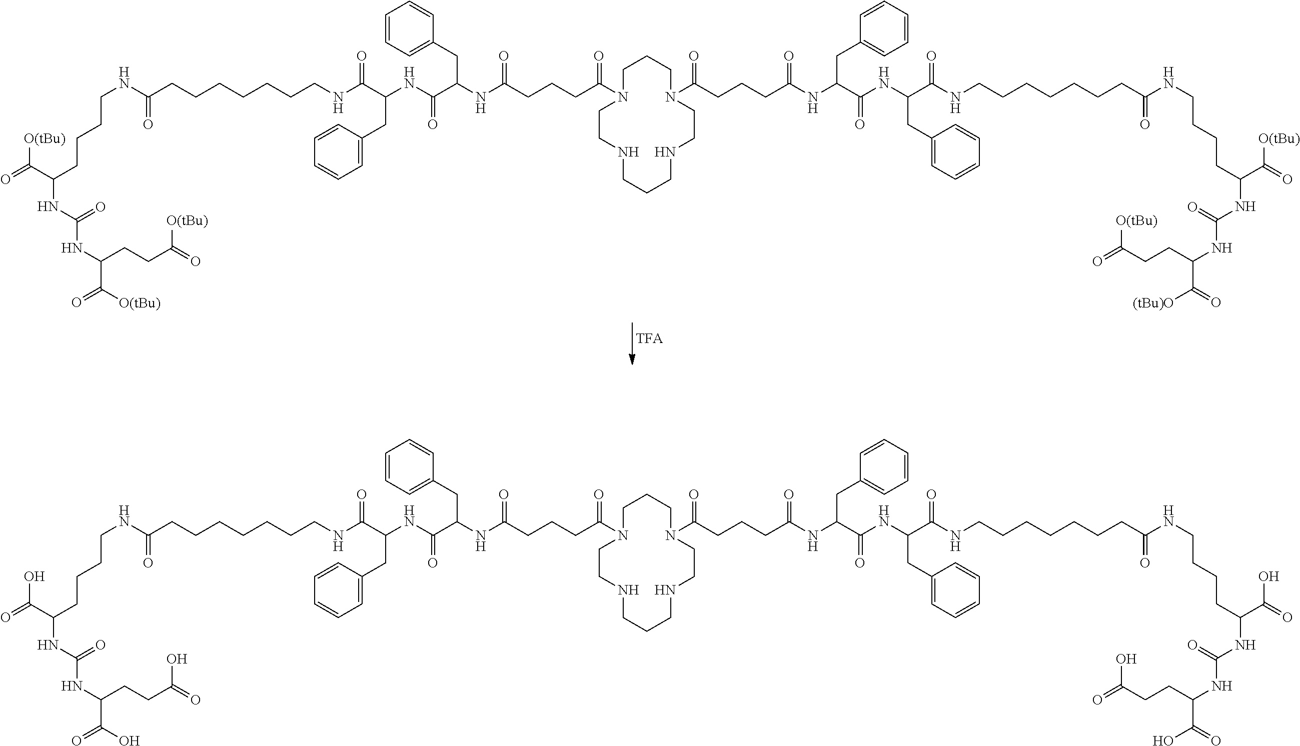

Scheme 2 also describes the coupling of two moieties of the linker-PSMA urea group with a metal chelator (specifically, a cyclam), however in this synthetic route, the amine group that participates in the coupling reaction is found on the metal chelator, while the carboxylic acid group is found on the linker-PSMA urea group. The reaction may also be performed under standard peptide coupling reaction conditions with a peptide coupling reaction and a base.

In certain embodiments, the present invention provides a process for preparing a compound of Formula (I) or a pharmaceutically acceptable salt thereof as described above, wherein the process includes one or more peptide coupling steps. The process may also include one or more protection and deprotection steps where appropriate. In other embodiments, the process for preparing a compound of Formula (I) includes a peptide coupling step performed under solution phase conditions with one or more peptide coupling reagents and one or more bases.

Variation in the structure of the chelator and subsequently the nature and the location of one or more functional groups will naturally dictate the synthetic route required to access the compounds of Formula (I). For example, where the terminal group on the metal chelator is oxygen-based, e.g. a carboxylic acid, then the protecting group (if required) and subsequent reaction should be compatible and allow for coupling to provide compounds of Formula (I).

The synthetic scheme depicted in Scheme 2 shows the coupling of the linker-PSMA urea group to two nitrogen atoms of cyclam, where the nitrogen atoms are adjacent to each other. The synthetic schemes disclosed herein encompass modifications that allow for the synthesis of regioisomers of the compounds of Formula (I). For example, the modification of the synthetic route and conditions in Scheme 2 will allow for the analogous compound of Formula (I) to be produced, where the linker-PSMA urea groups are attached to nitrogen atoms of cyclam that are non-adjacent.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

### EXAMPLES

The following examples are illustrative of the disclosure and should not be construed as limiting in any way the general nature of the disclosure of the description throughout this specification.

### Example 1. Synthesis of Compounds of the Invention

The various embodiments of the present invention may be prepared using the reaction routes and synthesis schemes as described below, employing the techniques available in the art using starting materials that are readily available. The preparation of particular compounds of the embodiments is described in detail in the following examples, but the artisan will recognize that the chemical reactions described may be readily adapted to prepare a number of other agents of the various embodiments. For example, the synthesis of non-exemplified compounds may be successfully performed by modifications apparent to those skilled in the art, e.g. by appropriately protecting interfering groups, by changing to other suitable reagents known in the art, or by making routine modifications of reaction conditions. A list of suitable protecting groups in organic synthesis can be found in T. W. Greene's Protective Groups in Organic Synthesis, 3rd Edition, John Wiley & Sons, 1991. Alternatively, other reactions disclosed herein or known in the art will be recognized as having applicability for preparing other compounds of the various embodiments. Reagents useful for synthesizing compounds may be obtained or prepared according to techniques known in the art.

### 1.1 Synthesis of PSMA (OtBu) 2; H-D-Phe-D-Phe-wCap-Lys-CO-Glu (OtBu)-OtBu

Fmoc-L-Lys (Dde)-OH was anchored to 2-chlorotrityl chloride resin and Fmoc-deprotected with 25% piperidine/DMF before coupling with a pre-activated solution of H-L-Glu (OtBu)-OtBu and carbonyldiimidazole (3 cq.) and DIPEA (6 eq.). The Dde protecting group was removed using hydrazine/DMF and the remaining peptide extending using standard solid-phase peptide synthesis methodology. The final Fmoc protecting group was removed and the peptide-resin treated with 20% trifluoroethanol/DCM overnight. The resin was filtered, the supernatant concentrated under reduced pressure and the resulting residue freeze-dried from 50% ACN/water. The PSMA (OtBu) 2 intermediate was obtained in 80% crude yield. MS-ESI: [M+H]⁺ calculated: 867.5, found: 867.5.

### 1.2 Synthesis of HBED-CC-bis(tert-butyl ester)

HBED-CC-bis(tert-butyl ester) was prepared as described in Makarem et al., Synlett 2008, “A Convenient Synthesis of HBED-CC-tris(tert-butyl ester)”. The method was modified to generate the symmetrical, bis-tert-butyl-protected product; HBED-CC-bis(tert-butyl ester). Briefly, 3-(4-hydroxyphenyl) propionic acid was methyl-ester protected and formylated over two steps to generate 3-(3-formyl-4-hydroxyphenyl) propionic acid methyl ester. This intermediate was reacted with ethylene diamine to form the symmetrical bis-imine and reduced to generate the symmetrical bis-amino derivative. Lastly, the amines were N-alkylated with tert-butyl bromoacetate and the methyl ester protecting groups hydrolysed under basic conditions. The crude product was purified by preparative RP-HPLC to provide the product at 98% purity. MS-ESI: [M+H]⁺ calculated: 645.3, found: 645.4.

### 1.3 Conjugation of DOTA-bis (PSMA)

Resin-bound PSMA (OtBu) 2 was prepared as described above. The peptide-resin was treated with a solution of DOTA (OtBu) 2 (1.5 eq.), HATU (1.5 cq.) and DIPEA (3 cq.) in DMF. After 5 h, the resin was drained, washed and treated with DIC and OxymaPure in DMF for 30 min. The resin was drained, washed once and immediately treated with a solution of PMSA(OtBu)₂ (2 cq.) and DIPEA (4 cq.) in DMF. After 4 h, the resin was drained, washed with DMF and DCM, and dried. The resin was treated with a TFA solution (89.5% TFA, 3% dithiothreitol, 2.5% triisopropylsilane, 5% water) and agitated for 4 h. The resin was filtered and the crude product precipitated from the supernatant with Et₂O. The precipitate was pelleted by centrifugation, the pellet dissolved in 50% ACN/water and freeze-dried to provide the crude product. The peptide was purified by preparative RP-HPLC to provide the product at 99% purity. MS-ESI: [M-2H]⁻² calculated: 937.5, found: 937.5, [M-3H]-3 calculated: 624.6, found: 625.5, [M-4H]⁻⁴ calculated: 468.2, found: 468.3.

### 1.4 Conjugation of HBED-CC-bis (PSMA)

Resin-bound PSMA (OtBu) 2 was prepared as described above. The peptide-resin was treated with a solution of HBED-CC-bis(tert-butyl ester) (1.5 eq., preparation described in Section 2), HATU (1.5 cq.) and DIPEA (3 eq.) in DMF. After 2 h, the resin was drained and immediately treated with a solution of PMSA(OtBu)₂ (2 eq.) and DIPEA (4 eq.) in DMF. After 4 h, the resin was drained, washed with DMF and DCM, and dried. The resin was treated with a TFA solution (92.5% TFA, 2.5% triisopropylsilane, 5% water) and agitated for 5 h. The resin was filtered and the crude product precipitated from the supernatant with Et₂O. The precipitate was pelleted by centrifugation, the pellet dissolved in 50% ACN/water and freeze-dried to provide the crude product. The peptide was purified by preparative RP-HPLC to provide the product at 98% purity. MS-ESI: [M+2H]⁺2 calculated: 1003.5, found: 1004.2, [M+3H]⁺3 calculated: 669.3, found: 669.6.

### 1.5 Conjugation of NOTA-bis (PSMA)

NOTA-(OH) 3 was activated with HBTU (2 eq.) and DIPEA (4 eq.) in DMF for 30 min. then treated with a solution of PSMA (OtBu) 2 (2 eq. relative to NOTA, preparation described in Section 1) and DIPEA (2 eq. relative to NOTA) in DMF. The reaction was stirred for 2 h, concentrated under reduced pressure and the resulting residue treated with a TFA solution (92.5% TFA, 2.5% triisopropylsilane, 5% water) with agitation for 4 h. The product was precipitated with Et₂O, pelleted by centrifugation, the pellet dissolved in 50% ACN/water and freeze-dried to provide the crude product. The peptide was purified by preparative RP-HPLC to provide the product at 99% purity. MS-ESI: [M+H]⁺ calculated: 1776.9, found: 1776.8, [M+2H]⁺2 calculated: 889.0, found: 889.0.

### Example 2. Radiolabelling Procedures

### 2.1 Preparation of ⁶⁸Ga-HBED-CC-bis (PSMA)

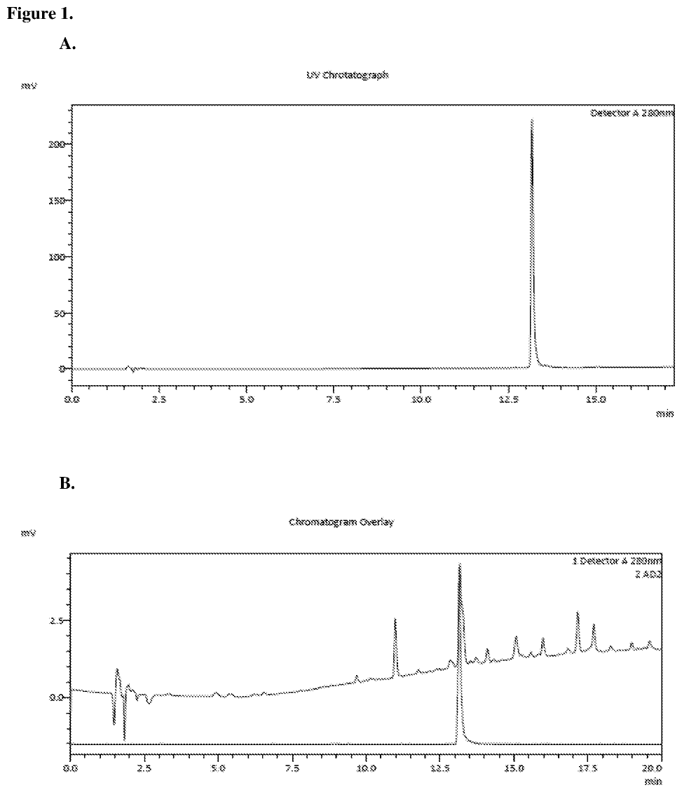

The HBED-bisPSMA compound was radiolabelled with ⁶⁸Ga at room temperature in aqueous solution of saline (0.9%, Baxter), HEPES (Sigma Aldrich), ethanol (Merck) and ascorbic acid (Sigma). The filtered solution was clear, colourless and particulate free and afforded ⁶⁸Ga-HBED-bisPSMA in >95% yield (radio HPLC, FIG. 1) and >95% radiochemical yield (radio ITLC).

### 2.2 Preparation of ⁶⁴Cu-DOTA-bis (PSMA) and ⁶⁴Cu-NOTA-bis (PSMA)

To an Eppendorf was added 120 μL of 0.25 M sodium acetate solution (pH 5.5; Huayi), 20 μL ethanol (Merck), 20 μL of 0.5% (w/v) gentisic acid (Huayi) in water and 80 μL (50 μg) of precursor compound (approximately 0.6 mg/mL). 64CuCl₂ (Austin Health) was volumetrically dispensed into the Eppendorf with a target activity of 200 MBq. The solution was incubated for 20 minutes at 45° C. Radiochemical purity of the resultant solutions of ⁶⁴Cu-DOTA-bis PSMA and ⁶⁴Cu-NOTA-bis PSMA were >99%.

### Example 3. Biodistribution Studies for Compounds of the Invention Complexed with ⁶⁸Ga of ⁶⁴Cu

### 3.1 Experimental Protocol

Thirty two male NSG mice were inoculated subcutaneously on the right flank with 6 million human PSMA expressing prostate cancer (LNCaP) cells in PBS: Matrigel (1:1). Mice were weighed and tumour volumes measured twice-weekly using electronic callipers. Tumour volume (mm³) was calculated as length×width×height×π/6.

51 days after inoculation 10 mice were randomised into Group 1 of the biodistribution study and then divided into two tumour volume-matched sub-groups (tumour volume range: 143-774 mm³, sub-group means 355 & 368 mm³). On the same day, HBED-CC-bis (PSMA) was labelled with ⁶⁸Ga and injected into mice of Group 1 via the tail vein.

On day 58, 8 mice were randomised into each of Group 2 and Group 3 of the biodistribution study and then both divided into two tumour volume-matched sub-groups (Group 2-tumour volume range: 36-510 mm³, sub-group means 231 & 237 mm³. Group 3-tumour volume range: 51-429 mm³, sub-group means 190 & 212 mm³). On the same day, NOTA-conjugated bis (PSMA) and DOTA-conjugated bis (PSMA) were labelled with ⁶⁴Cu and injected into the mice of Group 2 and Group 3, respectively.

For all three groups on their respective days, the two parallel sub-groups on n=5 (or 4) were harvested for tissue biodistribution and blood via cardiac puncture at 1 h or 3 h post-injection.

Biodistribution tissues were excised, weighed and counted using a Capintec (Captus 4000e) gamma counter. The data was analysed using Prism 9 for Windows (GraphPad).

### 3.2 Results

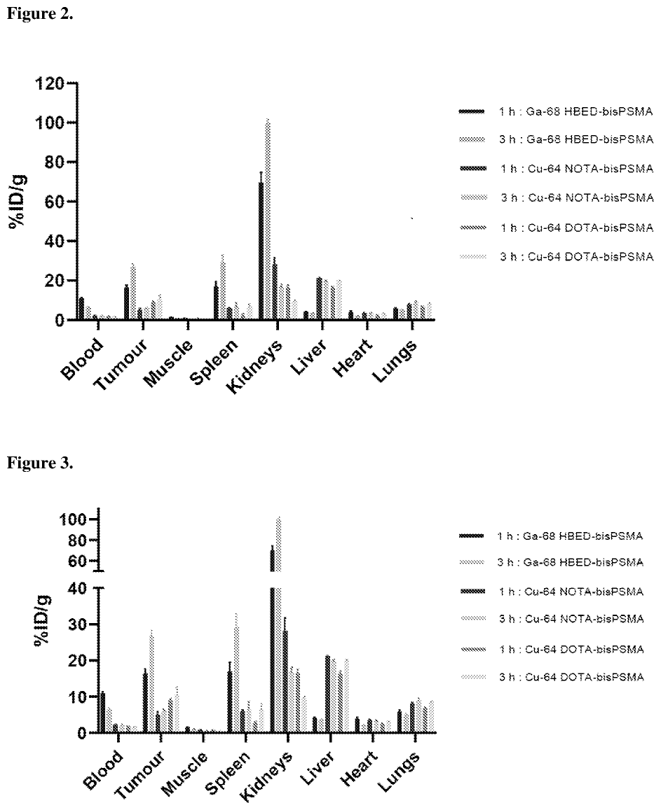

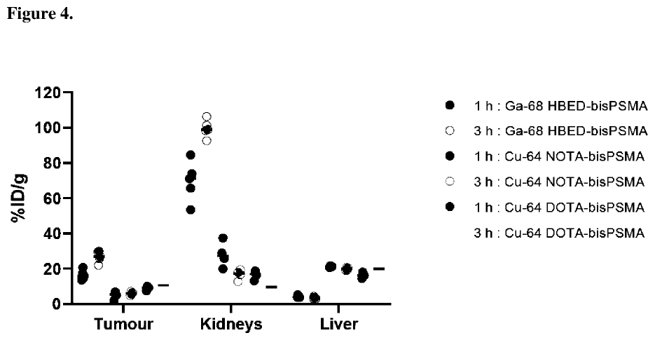

Biodistribution results are shown in FIGS. 2-4. Injected activities for ⁶⁸Ga-HBED-CC-bis (PSMA) were aimed at 6 MBq per mouse, with mean actual injected activity of 6.17 MBq. Injected activities for ⁶⁴Cu-DOTA-bis (PSMA) and ⁶⁴Cu-NOTA-bis (PSMA) were aimed at 5 MBq per mouse, with mean actual injected activity of 5.25 MBq and 5.04 MBq, respectively.

The radiolabelled PSMA ligands each showed distinct biodistribution profiles and kinetics between the gallium-radiolabelled HBED-CC-bis (PSMA) and the two copper-radiolabelled DOTA-bis (PSMA) and NOTA-bis (PSMA) compounds across the two timepoints. ⁶⁸Ga-HBED-CC-bis (PSMA) showed the highest tumour uptake. Mean % ID/g was consistent within the groups for each organ/time point.

1. A compound of Formula (I): 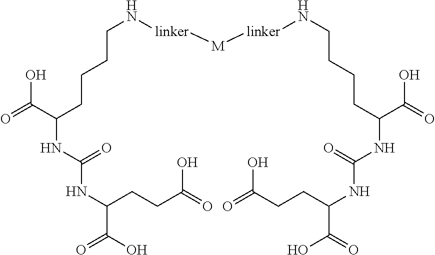
or a pharmaceutically acceptable salt, complex, isomer, solvate or prodrug thereof,
wherein M is a residue of a metal chelator selected from the group consisting of: 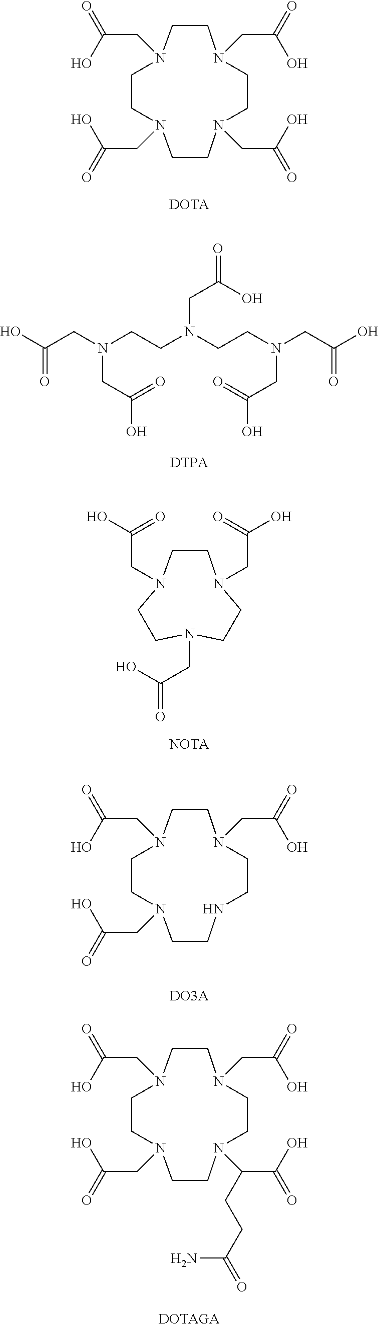 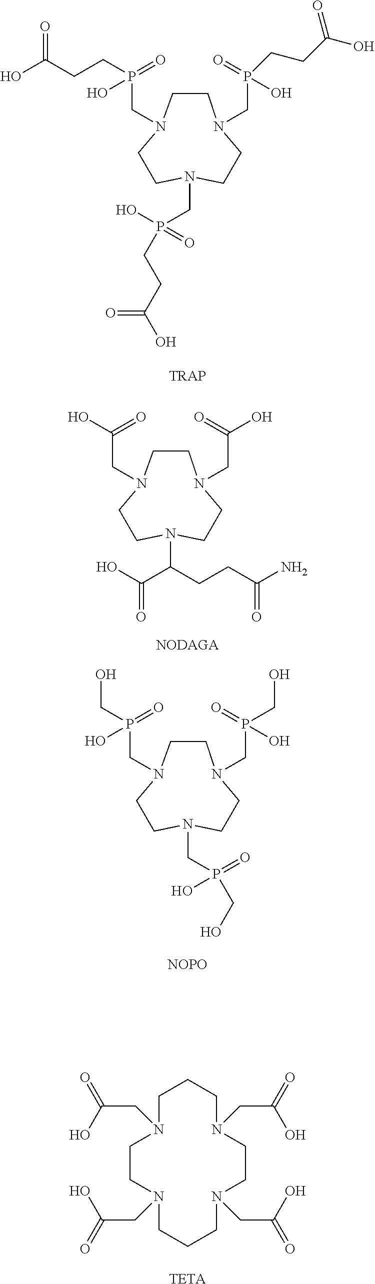 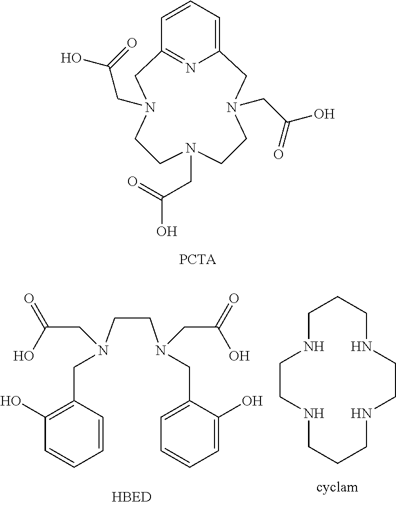
and wherein each linker may be the same or different and comprises one or more of the following groups or fragments thereof:

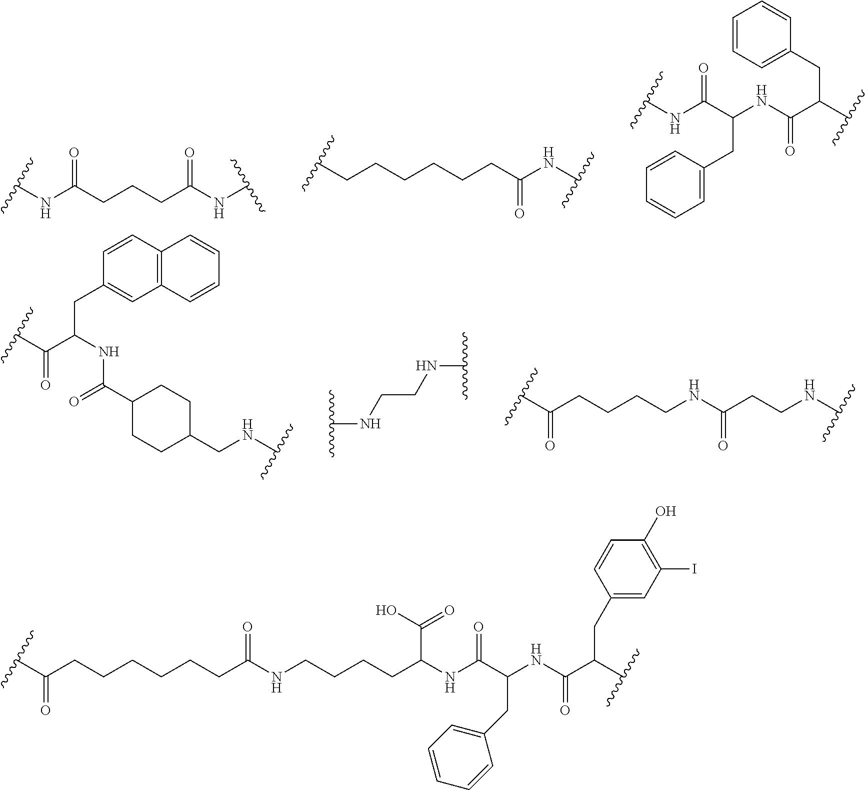

2. A compound of Formula (Ia): 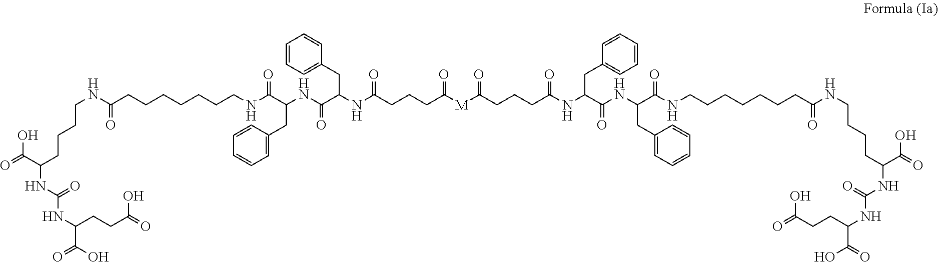
or a pharmaceutically acceptable salt, complex, isomer, solvate or prodrug thereof,
wherein M is a residue of a metal chelator selected from the group consisting of:

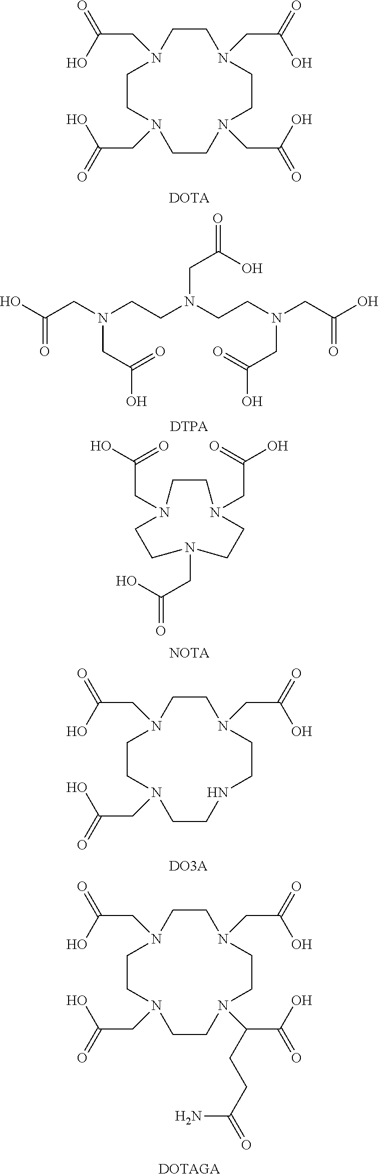

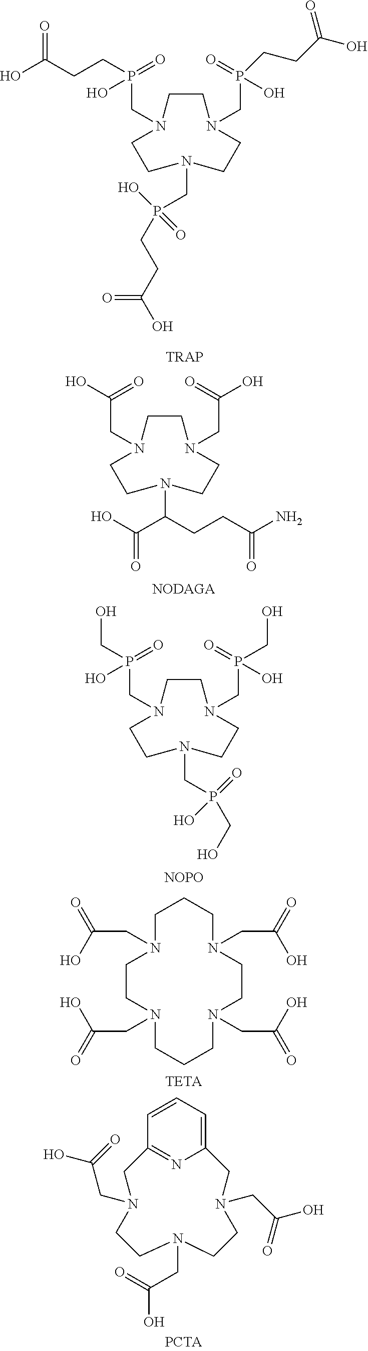

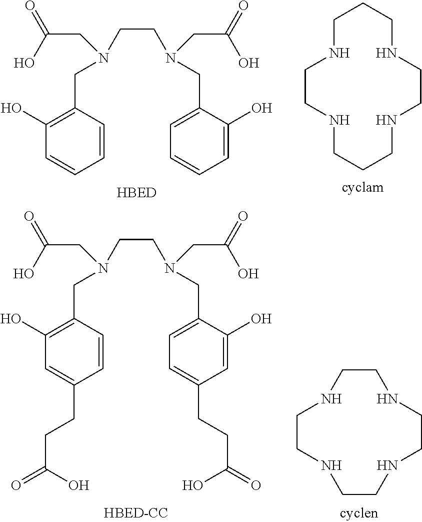 [SMILES: O=C(O)CN(CCN(CC(=O)O)Cc1ccccc1O)Cc1ccccc1O ; C1CNCCNCCCNCCNC1 ; O=C(O)CCc1ccc(CN(CCN(CC(=O)O)Cc2ccc(CCC(=O)O)cc2O)CC(=O)O)c(O)c1 ; C1CNCCNCCNCCN1]

3. A compound according to claim 1 or 2, wherein the metal chelator is complexed with an ion of metal selected from the consisting of Cu, Ga, Lu, F, Tc, In, Zr, Y, Rb, Ac, Rd, Re, Sm, Lu, Sc, Zr, Y, Ac, As, Ra and I.

4. A compound according to any one of claims 1 to 3, wherein the ion is a radionuclide.

5. A compound according to any one of claims 1 to 4, wherein the compound is complexed with a Cu radionuclide.

6. A compound according to claim 5, wherein the Cu radionuclide is selected from the group consisting of 60Cu, 61Cu, 62Cu, ⁶⁴Cu and ⁶⁷Cu.

7. A compound according to any one of claims 1 to 4, wherein the compound is complexed with a Ga radionuclide.

8. A compound according to claim 7, wherein the Ga radionuclide is ⁶⁸Ga.

9. A compound according to any one of claims 1 to 4, wherein the compound is complexed with a Lu radionuclide.

10. A compound according to claim 9, wherein the Lu radionuclide is ¹⁷⁷Lu.

11. A compound according to any one of claims 1 to 4, wherein the compound is complexed with a radionuclide selected from the group consisting of ⁹⁰Y, ¹¹¹In, ¹⁷⁷Lu, ¹⁸⁸Re, ²¹¹As, ²¹²Pb and ²²⁵Ac.

12. A compound according to claim 1, wherein the compound has one of the following structures:

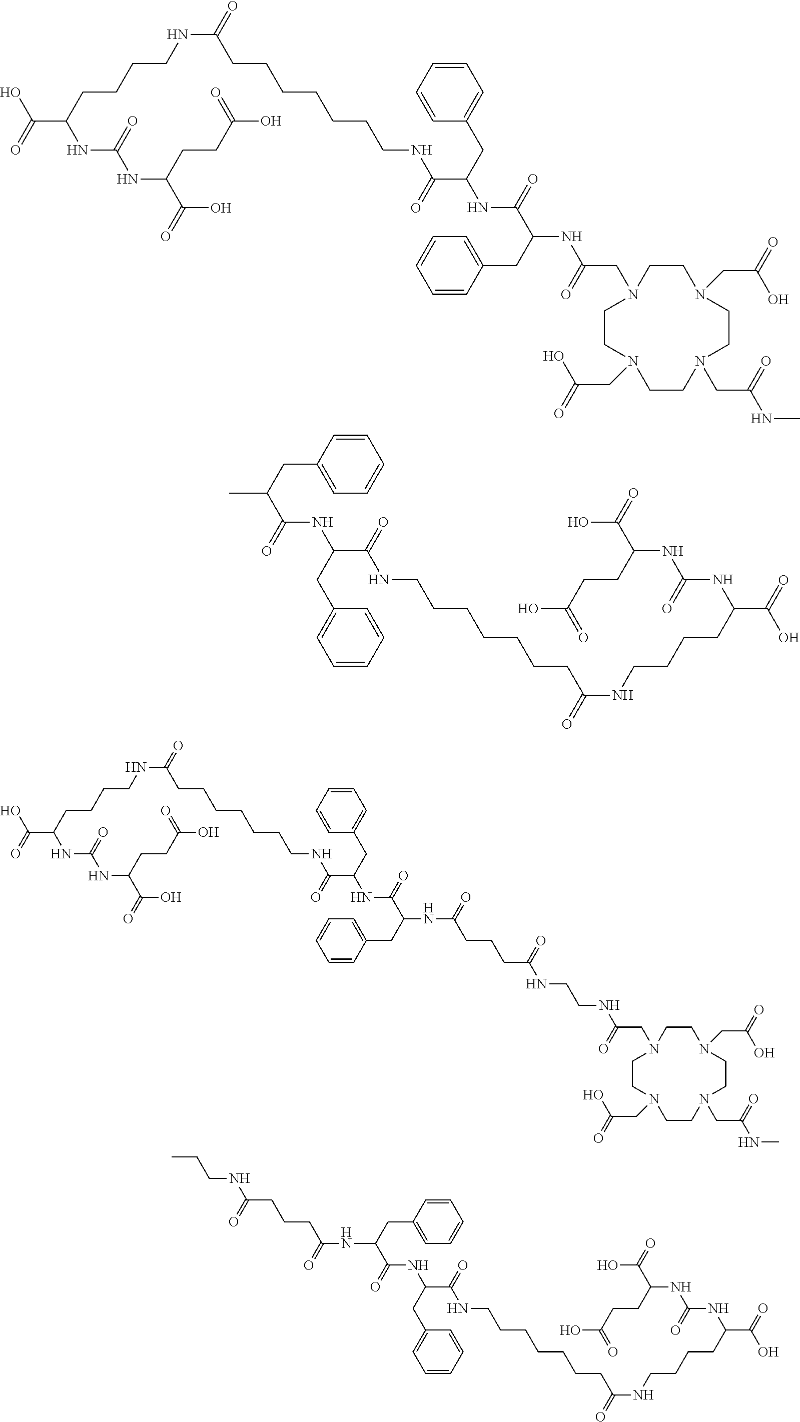

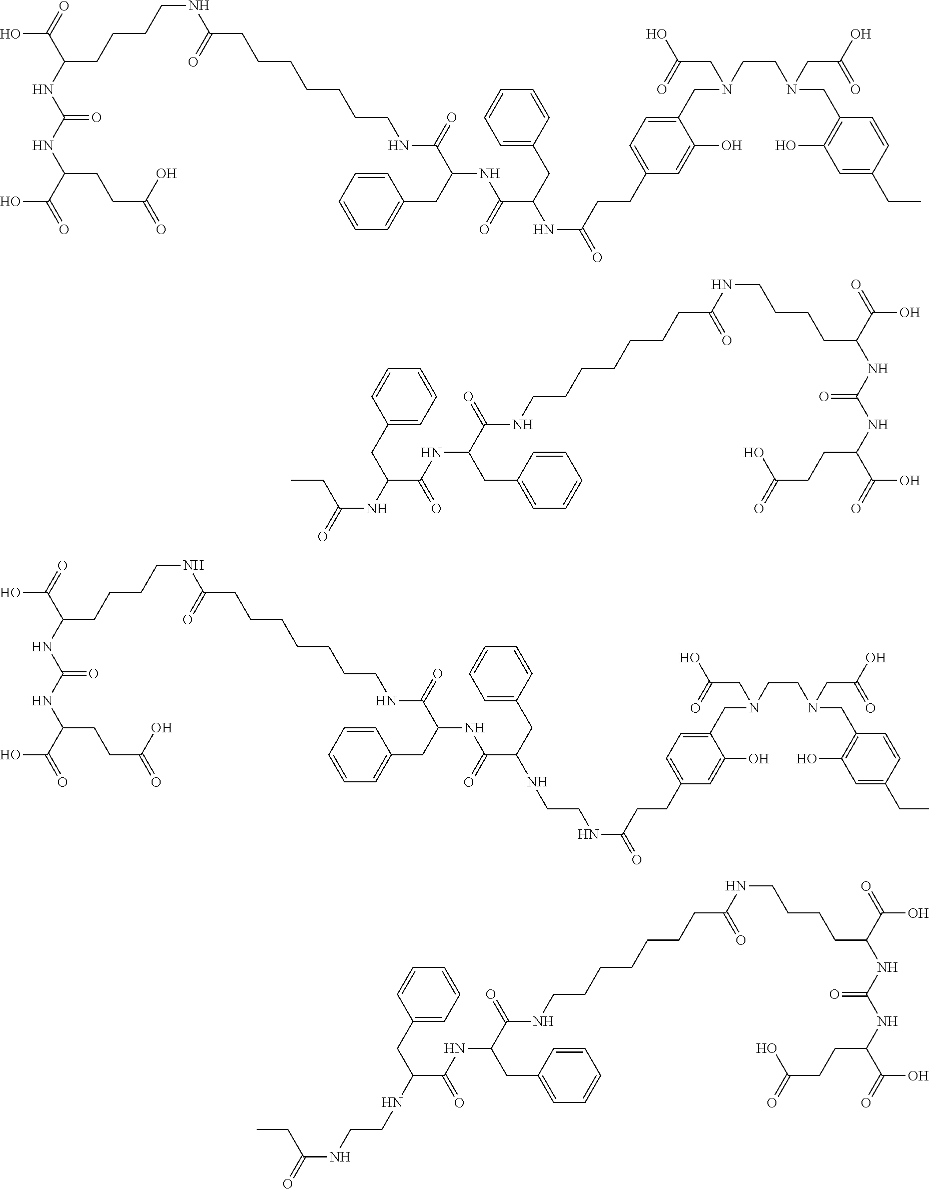

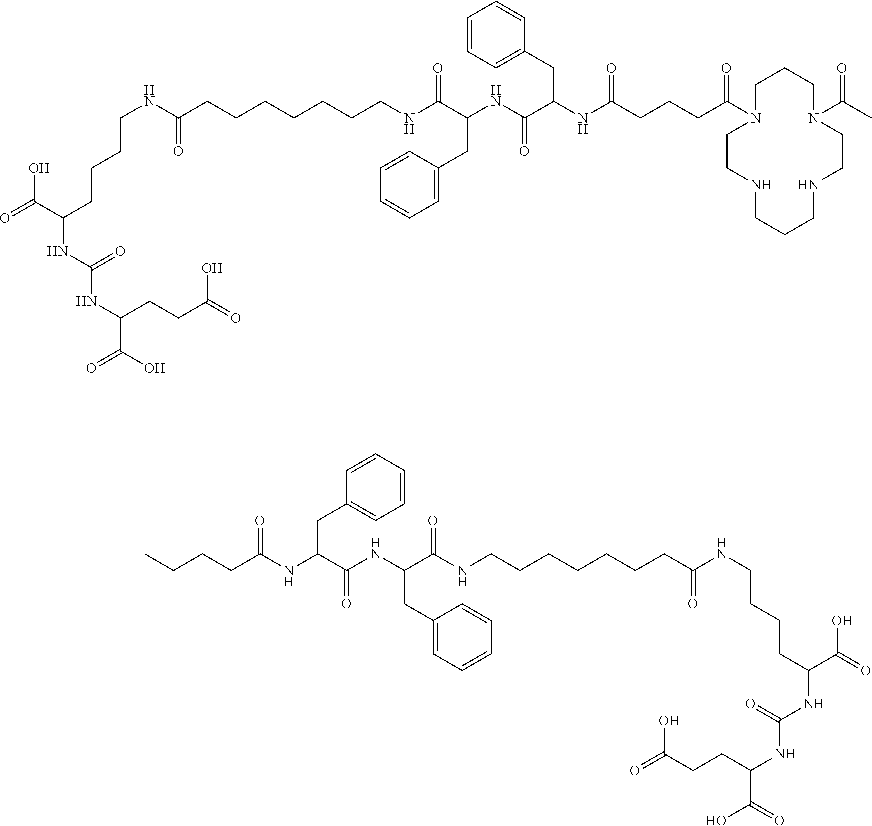

13. A compound according to claim 12, wherein the compound is complexed with a radionuclide.

14. A composition comprising a compound as defined in any one of claims 1 to 13 and one or more pharmaceutically acceptable excipients.

15. A method for the radioimaging of a cancer in a subject in need thereof, the method comprising administering to the subject a compound according to any one of claims 4 to 11 and 13.

16. A method according to claim 15, wherein the cancer is characterised by the overexpression of PSMA.

17. A method according to claim 15 or 16, wherein the cancer is prostate cancer.

18. A method for the treatment of a cancer in a subject in need thereof, the method comprising administering to the subject a compound according to any one of claims 4 to 11 and 13.

19. A method according to claim 18, wherein the cancer is characterised by the overexpression of PSMA.

20. A method according to claim 17 or 18, wherein the cancer is prostate cancer.

21. Use of a compound according to any one of claims 1 to 13 in the manufacture of a medicament for radioimaging a cancer.

22. Use according to claim 21, wherein the cancer is characterised by the overexpression of PSMA.

23. Use according to claim 21 or 22, wherein the cancer is prostate cancer.

24. Use of a compound according to any one of claims 1 to 13 in the manufacture of a medicament for treating a cancer.

25. Use according to claim 24, wherein the cancer is characterised by the overexpression of PSMA.

26. Use according to claim 24 or 25, wherein the cancer is prostate cancer.
